(19) United States
(12) Patent Application Publication
(10) Pub. No.: US 2026/0265297 A1
(43) Pub. Date: Sep. 10, 2026

(54) UNNATURAL PEPTIDE ASSEMBLIES, AND METHODS OF USING AND MAKING THE SAME

(71) Applicant: Brandeis University, Waltham, MA (US)

(72) Inventors: Qiuxin Zhang, Lexington, MA (US); Bing Xu, Newton, MA (US)

(21) Appl. No.: 19/557,324

(22) Filed: Mar. 5, 2026

Related U.S. Application Data
(60) Provisional application No. 63/767,636, filed on Mar. 6, 2025.

(51) Int. Cl. C07K 5/06; A61K 38/00; C07K 5/08

(52) U.S. Cl. CPC C07K 5/06; C07K 5/08; A61K 38/00

(57) ABSTRACT

The present disclosure relates to unnatural peptide assemblies designed for therapeutic modulation of membrane biophysics, particularly for targeting cancer cells. Alterations in lipid composition and biophysical properties of cellular membranes, along with disrupted cell signaling, are linked to various diseases, including cancer. Cholesterol, a critical component for membrane integrity and cancer progression, represents a promising therapeutic target. The disclosed unnatural peptide assemblies are capable of depleting cholesterol from membranes, thereby inhibiting cancer cell viability and proliferation. Additionally, pharmaceutical compositions comprising these peptide assemblies, or their pharmaceutically acceptable derivatives, are described, offering a therapeutic approach to cancer treatment.

### CROSS-REFERENCE TO RELATED APPLICATION

This application claims the benefit of and priority to U.S. Provisional Patent Application No. 63/767,636, filed on Mar. 6, 2025, which application is incorporated by reference herein in its entirety.

### STATEMENT OF GOVERNMENT SUPPORT

This invention was made with government support under CA142746 awarded by the National Institutes of Health; and under DMR2011846 awarded by the National Science Foundation. The government has certain rights in the invention.

### BACKGROUND OF THE INVENTION

### 1. Field of the Invention

This invention is directed to unnatural peptides, and more specifically to unnatural peptide assemblies that can rapidly deplete cholesterol and potentially inhibit cancer cells.

### 2. Brief Description of the Related Art

Following the proposal of the classical membrane fluid mosaic model by Singer and Nicholson, numerous studies have delved into the refinement of its structure and exploration of associated functions. A plasma membrane houses hundreds of lipids and provides a heterogeneous cell boundary from the extracellular matrix. The same sequestration strategy is employed to form various types of cell membranes, dividing the interior of cells into multiple membrane-bound organelles. The heterogeneous distribution of lipids in cell membranes forms microdomains, which proteins interact with to render complicated physiological properties to the lipid bilayer through cell signaling.

Alteration in lipid composition and biophysical properties of membranes and, ultimately, change in cell signaling are associated with a variety of diseases, such as cancer, neurodegenerative disorders, cardiovascular pathologies, etc. Accordingly, the strategy of regulating membrane composition and structure through pharmaceutical approaches, termed membrane lipid therapy (MILT), has been developed to manipulate key protein interactions and normalize pathological signaling cascades. However, most of the agents for MLT are small molecules designed solely for a certain factor regulating membrane lipids directly or indirectly, which greatly limits its selectivity, bioavailability, functionality, and finally its efficacy. Therefore, there is an unmet need for a versatile membrane modulation platform.

Cholesterol is crucial for both membrane integrity and cancer development. Studies have shown its role in promoting colon, breast, and prostate cancers. This makes it an attractive target for therapy. For example, a cholesterol synthesis inhibitor can induce apoptosis in prostate cancer cells by targeting their elevated cholesterol synthesis. Additionally, MβCD, a millimolar cholesterol-depleting agent, augments the efficacy of tamoxifen in breast cancer and melanoma cells due to higher expression of cholesterol-rich microdomains in tumor cells. Intracellular cholesterol accumulation was reported to enhance cancer aggressiveness. Depleting cholesteryl ester storage by blocking cholesterol esterification is being explored in various cancers. Cholesterol was conjugated with peptides and acted as a substrate of enzymatic noncovalent synthesis for killing drug-resistant cancer cells through in situ self-assembly.

Cholesterol-rich membranes are critically involved in cancer initiation and progression, highlighting the need for innovative strategies to target these membranes for cancer inhibition.

### SUMMARY OF THE INVENTION

The present disclosure describes unnatural peptide assemblies capable of depleting cholesterol and inhibiting cancer cells.

More specifically, the present disclosure describes novel, unnatural peptide assemblies with remarkable abilities of rapid cholesterol depletion in cancer cells and potent cytotoxicity against malignant cancer cells. The amine group of bb-NBD likely contributes to displacing cholesterol due to stronger lipid head affinity. Crucially, this work highlights the versatility of peptide assemblies in targeting cholesterol-rich membranes and hindering cancer cell proliferation. bb-NBD showed selectivity for HeLa cells over PC3 and HepG2, which correlates with higher LDLR expression in PC3 and HepG2. Lovastatin treatment further enhanced bb-NBD's selectivity for PC3 over HepG2 cells. This approach paves the way for further therapeutic development by integrating unnatural amino acids with distinct cancer-targeting mechanisms, such as redox reactions or enzymatic processes, to target cell membranes and modulate aberrant lipid metabolism in cells.

In one aspect, disclosed herein are unnatural peptide assemblies, which can modulate membrane biophysics for therapeutic purposes. In some embodiments, the unnatural peptide assemblies disclosed herein can manipulate the membrane biophysics of cancer cells by rapidly depleting cholesterol.

In some embodiments, the unnatural peptide assembly is of Formula I, or a tautomer, a pharmaceutically acceptable salt, a solvate, a hydrate, a clathrate or a polymorph thereof:

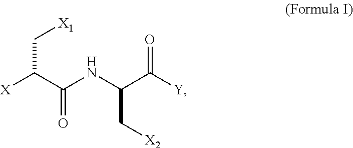

wherein
X is selected from NH₂, NHX₃, OH,

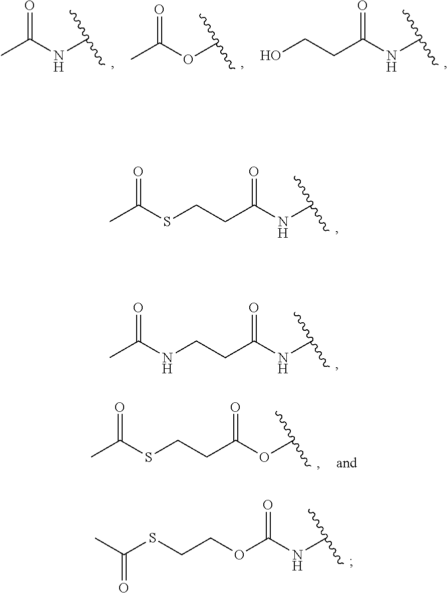

wherein X₁ is selected from biphenyl and phenyl; wherein X₂ is selected from biphenyl and phenyl; and wherein X₃ is selected from Glycine, D-Alanine, D-Serine, D-Lysine,

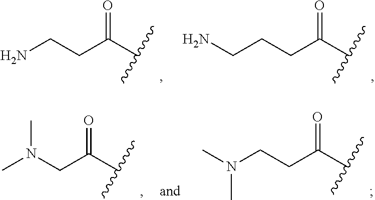

wherein Y is selected from

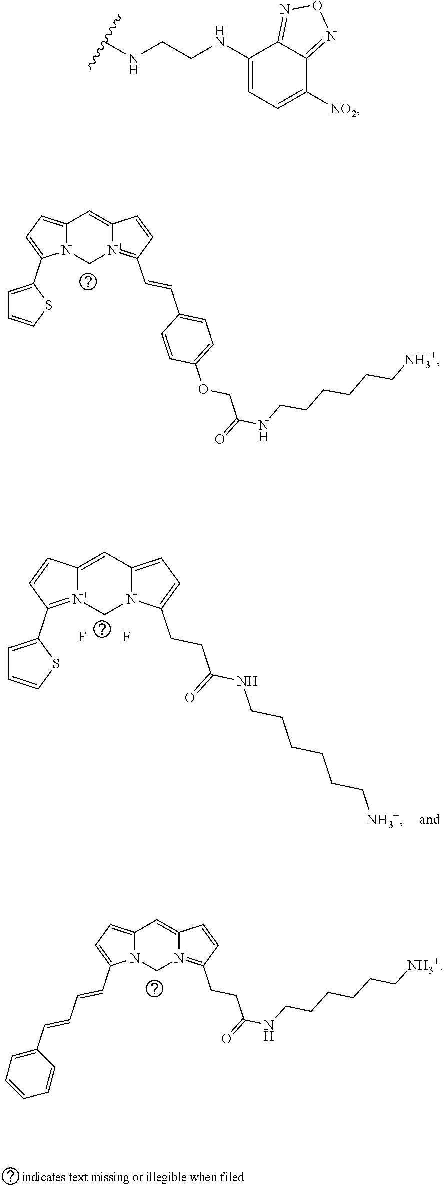

In some embodiments, X is NH₂, X₁ is biphenyl, X₂ is biphenyl, and Y is

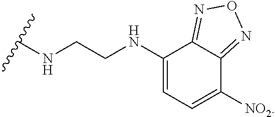 [SMILES: *NCCNc1ccc([N+](=O)[O-])c2nonc12]

In some embodiments, X is

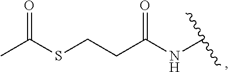 [SMILES: *NC(=O)CCSC(C)=O]

X₁ is biphenyl, X₂ is biphenyl, and Y is

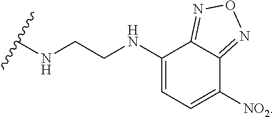 [SMILES: *NCCNc1ccc([N+](=O)[O-])c2nonc12]

In some embodiments, X is NHX₃ and X₃ is Glycine; X₁ is biphenyl and X₂ is biphenyl; and Y is

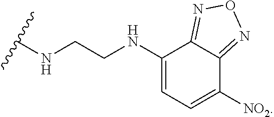 [SMILES: *NCCNc1ccc([N+](=O)[O-])c2nonc12]

In some embodiments, the unnatural peptide assembly is of Formula II, or a tautomer, a pharmaceutically acceptable salt, a solvate, a hydrate, a clathrate or a polymorph thereof:

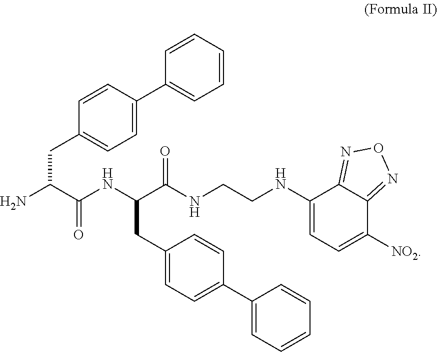 [SMILES: N[C@H](Cc1ccc(-c2ccccc2)cc1)C(=O)N[C@H](Cc1ccc(-c2ccccc2)cc1)C(=O)NCCNc1ccc([N+](=O)[O-])c2nonc12]

In some embodiments, the unnatural peptide assembly is of Formula III, or a tautomer, pharmaceutically acceptable salt, solvate, hydrate, clathrate or polymorph thereof.

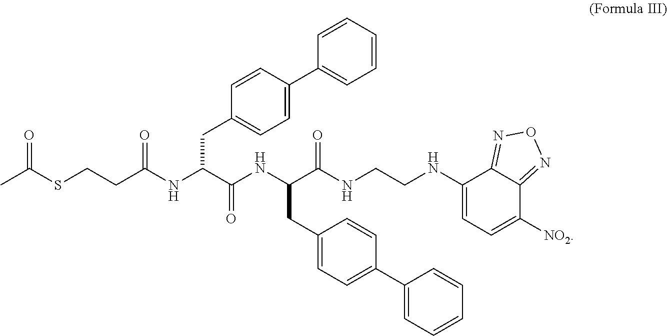 [SMILES: CC(=O)SCCC(=O)N[C@H](Cc1ccc(-c2ccccc2)cc1)C(=O)N[C@H](Cc1ccc(-c2ccccc2)cc1)C(=O)NCCNc1ccc([N+](=O)[O-])c2nonc12]

In some embodiments, the unnatural peptide assembly is of Formula IV, or a tautomer, pharmaceutically acceptable salt, solvate, hydrate, clathrate or polymorph thereof:

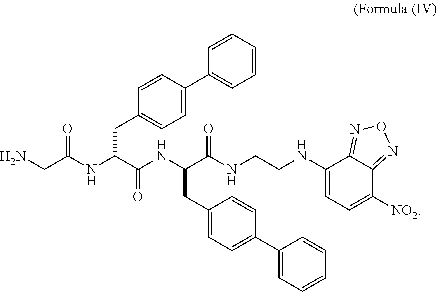 [SMILES: NCC(=O)N[C@H](Cc1ccc(-c2ccccc2)cc1)C(=O)N[C@H](Cc1ccc(-c2ccccc2)cc1)C(=O)NCCNc1ccc([N+](=O)[O-])c2nonc12]

In some embodiments, the unnatural peptide assembly contains an unnatural lipophilic motif, for example, D-biphenylalanine-D-biphenylalanine (bb), and a fluorophore.

In some embodiments, the unnatural peptide assembly contains an unnatural lipophilic motif, for example, D-biphenylalanine-D-biphenylalanine (bb), for self-assembly, and a fluorophore for imaging.

In some embodiments, the fluorophore is nitrobenzoxadiazole (NBD).

In some embodiments, the unnatural peptide assembly contains both D-biphenylalanine-D-biphenylalanine (bb) and nitrobenzoxadiazole (NBD) to form bb-NBD.

In one aspect, disclosed herein is a pharmaceutical composition that comprises an unnatural peptide assembly as taught and disclosed herein, or a tautomer, a pharmaceutically acceptable salt, a solvate, a hydrate, a clathrate or a polymorph thereof; and one or more pharmaceutically acceptable excipients or carriers.

In some embodiments, the pharmaceutical further comprises an additional therapeutic agent, which is selected from alkylating agents, cytotoxic antibiotics, anti-metabolites, anti-microtubule agents, histone deacetylase inhibitors, kinase inhibitors, topoisomerase I inhibitors, topoisomerase II-targeting agents, DNA or RNA synthesis inhibitors, protein synthesis inhibitors, cell growth and differentiation regulators, cell proliferation inhibitors, apoptosis inducers, agonists/activators of tumor necrosis factor receptor superfamily member 4, agonists/activators of TNFRSF member 5, agonists/activators of TNFRSF member 9, agonists/activators of TNFRSF member 18, agonists/activators of toll-like receptors, cytokines and hormones, inhibitors of programmed cell death 1 (PD-1) receptor, inhibitors of cytotoxic T lymphocyte-associated protein 4, inhibitors of killer cell immunoglobulin-like receptors, and inhibitors of lymphocyte activation gene 3 (LAG-3) receptor.

In one aspect, disclosed herein is a method of treating a tumor or cancer by administering to a subject in need thereof an amount of an unnatural peptide assembly as taught and disclosed herein, or a tautomer, a pharmaceutically acceptable salt, a solvate, a hydrate, a clathrate or a polymorph thereof, effective to treat a tumor or cancer.

In some embodiments, the tumor or cancer is selected from solid tumors, head and neck tumors and cancers (e.g., oral squamous cell carcinoma, esophageal squamous cell carcinoma, nasopharyngeal carcinoma and papillary thyroid cancer), tumors and cancers of the CNS (e.g., glioblastoma, glioma and neuroblastoma), lung cancer (e.g., non-small cell lung cancer), gynecological cancers (e.g., breast cancer, ovarian cancer [e.g., high-grade serous carcinoma], and uterine cancer [e.g., cervical cancer and endometrial cancer]), cancers of the gastrointestinal tract (e.g., stomach cancer [e.g., gastric carcinoma] and colorectal cancer), liver cancer (e.g., hepatocellular carcinoma and intrahepatic cholangiocarcinoma), pancreatic cancer (e.g., pancreatic adenocarcinoma), urological cancers (e.g., kidney cancer [e.g., renal clear cell carcinoma and renal papillary cell carcinoma], bladder cancer and prostate cancer), skin cancer (e.g., melanoma), sarcomas, and hematological and lymphoid cancers (e.g., leukemias and lymphomas [e.g., non-Hodgkin lymphomas such as diffuse large B-cell lymphoma]).

In some embodiments, the unnatural peptide assembly is administered orally.

In some embodiments, the unnatural peptide assembly is administered intratumorally, parenterally, intravenously, subcutaneously, or intramuscularly.

In one aspect, disclosed herein is a method of inhibiting the proliferation or growth of cancer cells by contacting cancer cells, with an unnatural peptide assembly as taught and disclosed herein, or a tautomer, a pharmaceutically acceptable salt, a solvate, a hydrate, a clathrate or a polymorph thereof. In some embodiments, the contacting occurs in vivo, ex vivo or in vitro.

In some embodiments, the cancer cells are cervical cancer cells, prostate cancer cells, or liver cancer cells.

### BRIEF DESCRIPTION OF THE DRAWINGS

A better understanding of features and advantages of the present disclosure will be obtained by reference to the following detailed description, which sets forth illustrative embodiments of the disclosure, and the accompanying drawings.

FIG. 1A and FIG. 1B show the confocal microscopy of HeLa cells treated with bb-NBD (FIG. 1A: dose response, FIG. 1B: 10 μM time-lapse). (Bar=20 μm).

FIGS. 2A-G show that HeLa cells treated with 2 μM of bb-NBD. FIG. 2A: Endocytosis inhibitor effects (quantification). FIGS. 2B and 2D: Images with MβCD/Dynasore pretreatment (CLSM). FIG. 2C: MβCD dose dependence (quantification). FIG. 2E: Instantaneous treatment with/without MβCD (CLSM with CellMask stain). FIGS. 2F and 2G: Cholesterol depletion with pre-treatment (CLSM & quantification). (Scale bar=20 μm).

FIGS. 3A-C show that HeLa cells treated with bb-NBD and colocalization analysis of bb-NBD vs. trackers. FIG. 3A: HeLa cells stained with trackers, treated with bb-NBD (2 μM, 20 min), and imaged (CLSM). FIG. 3B: Time-lapse of ER-stained cells treated with bb-NBD (2 μM, CLSM). FIG. 3C: Colocalization analysis (Manders' coefficients) of bb-NBD vs. trackers. (Scale bar=20 m).

FIGS. 4A-E show the cell viability of HeLa and PC3 cells treated with bb-NBD with or with-out the addition of Lovastatin. The cell viability of (FIG. 4A) HeLa and (FIG. 4B) PC3 cells treated with bb-NBD and (FIG. 4C) the IC50 values of bb-NBD against HeLa and PC3 cells. Cell viability of HeLa cells treated (FIG. 4D) with bb-NBD (24 h), with or with-out the addition of Lovastatin (20 μM) and (FIG. 4E) with bb-NBD (1 h), with or without MβCD (5 mM, 30 min) pretreatment.

FIGS. 5A-D show the rapid cholesterol depletion from membranes by bb-NBD and the structures of bb-NBD and its analogs. FIG. 5A: rapid cholesterol depletion from membranes by bb-NBD. FIG. 5B: structure of bb-NBD. FIG. 5C: structure of NBD-bb. FIG. 5D: structure of ff-NBD.

FIGS. 6A and B show IC₅₀ values of an unnatural peptide assembly against different cancer cell lines in 24 h. FIG. 6A: Chemical structures of additional unnatural peptides. FIG. 6B: IC50 values of peptide 6 against different cancer cell lines in 24 h.

FIGS. 7A and B shows the cytotoxicity of an unnatural peptide assembly with or without NMT inhibitors against HeLa cells in 24 h. FIG. 7A: Chemical structures of additional unnatural peptides. FIG. 7B: Cytotoxicity of peptide 9 with or without NMT inhibitors against HeLa cells in 24 h.

FIG. 8: An exemplary synthetic scheme of unnatural peptides and related derivatives of the present disclosure.

### DETAILED DESCRIPTION OF THE INVENTION

### General Disclosure

To facilitate an understanding of the present invention, a number of terms and phrases are defined below.

Unless defined otherwise, all technical and scientific terms used herein have the same meaning as commonly understood by one of ordinary skill in the art to which this invention belongs. The abbreviations used herein have their conventional meaning within the chemical and biological arts. The chemical structures and formulae set forth herein are constructed according to the standard rules of chemical valency known in the chemical arts.

While various embodiments of the present disclosure are described herein, it will be obvious to those skilled in the art that such embodiments are provided by way of example only. Numerous modifications and changes to, and variations and substitutions of, the embodiments described herein will be apparent to those skilled in the art without departing from the disclosure. It is understood that various alternatives to the embodiments described herein can be employed in practicing the disclosure. It is also understood that every embodiment of the disclosure can optionally be combined with any one or more of the other embodiments described herein which are consistent with that embodiment.

Where a combination is disclosed, it is understood that each possible sub-combination of the elements of that combination is also disclosed. Conversely, where different elements or groups of elements are individually disclosed, combinations thereof are also disclosed.

Where elements are presented in list format or as alternative members of a group (e.g., a Markush group), it is understood that each possible subgroup of the elements is also disclosed, and any one or more elements can be removed from the list or group.

Where a range of numerical values is recited, it is understood that the endpoints and each intervening integer value and each fraction thereof, as well as each subrange, between the recited endpoints (upper and lower limits) of that range are specifically disclosed. The endpoints of all ranges are included within the range and are independently combinable. Where a value has an inherent limit, that inherent limit is specifically disclosed. Where a value is explicitly recited, it is understood that values which are about the same as the recited value are specifically disclosed.

It is also understood that, unless clearly indicated to the contrary, in any method described or claimed herein that includes more than one act or step, the order of the acts or steps of the method is not necessarily limited to the order in which the acts or steps of the method are recited, but the disclosure encompasses embodiments in which the order is so limited.

It is further understood that, in general, where an embodiment in the description or the claims is referred to as comprising one or more features, the disclosure also encompasses embodiments that consist of, or consist essentially of, such feature(s).

It is also understood that any embodiment of the disclosure, e.g., any embodiment or compound found within the prior art, can be explicitly excluded from the claims, regardless of whether or not the specific exclusion is recited in the specification.

In addition, it is understood that any functional language used in any claims shall not be construed as “means-plus-function” language under 35 U.S.C. § 112(f), unless specifically expressed as such by use of the term “means for” or “step(s) for” in a claim.

It is further understood that the present disclosure encompasses analogs, derivatives, prodrugs, metabolites, salts, solvates, hydrates, clathrates and polymorphs of all the compounds/substances disclosed herein, as appropriate. The specific recitation of “analogs,”,” “derivatives,”,” “prodrugs,” “metabolites,” “salts,” “solvates,” “hydrates,” “clathrates” or “polymorphs” with respect to a compound/substance or a group of compounds/substances in certain instances of the disclosure shall not be interpreted as an intended omission of any of these forms in other instances of the disclosure where the compound/substance or the group of compounds/substances is mentioned or shown without recitation of any of these forms, unless stated otherwise or the context clearly indicates otherwise.

It is also understood that the present disclosure encompasses all possible tautomers, all possible regioisomers, and all possible stereoisomers, including both enantiomers and all possible diastereomers in substantially pure form and mixtures of both enantiomers in any ratio (including a racemic mixture of enantiomers) and mixtures of two or more diastereomers in any ratio, of the compounds/substances described herein as appropriate, and not only the specific tautomers, regioisomers and stereoisomers as indicated by drawn structure or nomenclature. Some embodiments of the disclosure relate to the specific tautomers, regioisomers and stereoisomers indicated by drawn structure or nomenclature. The specific recitation of the phrase “or tautomers thereof,” “or regioisomers thereof,” “or stereoisomers thereof” or the like with respect to a compound/substance or a group of compounds/substances in certain instances of the disclosure shall not be interpreted as an intended omission of any of the other possible tautomers, regioisomers and stereoisomers of the compound/substance or the group of compounds/substances in other instances of the disclosure where the term “compound” or the like is used, or where the compound/substance or the group of compounds/substances is mentioned or shown, without recitation of the phrase “or tautomers thereof,” “or regioisomers thereof,” “or stereoisomers thereof” or the like, unless stated otherwise or the context clearly indicates otherwise.

Headings are included herein for reference and to aid in locating certain sections. Headings are not intended to limit the scope of the embodiments and concepts described in the sections under those headings, and those embodiments and concepts may have applicability in other sections throughout the entire disclosure.

All patent literature and all non-patent literature cited herein are incorporated herein by reference in their entirety to the same extent as if each patent literature or non-patent literature were specifically and individually indicated to be incorporated herein by reference in its entirety.

### Definitions

Unless defined otherwise or clearly indicated otherwise by their use herein, all technical and scientific terms used herein have the same meaning as commonly understood by those of ordinary skill in the art to which this application belongs.

As used in the specification and the claims, the indefinite articles “a” and “an” and the definite article “the” can include plural referents as well as singular referents unless specifically stated otherwise or the context clearly indicates otherwise. By way of example, “an element” means one element or more than one element. By way of further example “an analogue” means one analogue or more than one analogue.

The terms “or/and” and “and/or” mean “either . . . or . . . , or both . . . and . . . ” when referring to two elements, and mean “either . . . , . . . or . . . , or any combination or all thereof” when referring to three or more elements. As an example, the phrase “A or/and B” means “either A or B, or both A and B,” and the phrase “A, B or/and C” means “either A, B or C, or any combination or all thereof”.

As used in the specification and the claims, all transitional terms such as “comprising,” “containing,” “having,” “including,” “possessing,” “holding,” “carrying,” “bearing,” “composed of,” “characterized by” including grammatical equivalents and the like are open-ended and inclusive, that is, mean including but not limited to and do not exclude additional, unrecited element(s) or method step(s).

The term “exemplary” as used herein means “serving as an example, instance or illustration”. Any embodiment or feature characterized herein as “exemplary” should not be construed as preferred or advantageous over other embodiments or features.

The term “about” or “approximately” means an acceptable error for a particular value as determined by one of ordinary skill in the art, which depends in part on how the value is measured or determined. In certain embodiments, the term “about” or “approximately” means within one standard deviation. In some embodiments, when no particular margin of error (e.g., a standard deviation to a mean value given in a chart or table of data) is recited, the term “about” or “approximately” means that range which would encompass the recited value and the range which would be included by rounding up or down to the recited value as well, taking into account significant figures. In certain embodiments, the term “about” or “approximately” means within ±10% or 5% of the specified value. Whenever the term “about” or “approximately” precedes the first numerical value in a series of two or more numerical values or in a series of two or more ranges of numerical values, the term “about” or “approximately” applies to each one of the numerical values in that series of numerical values or in that series of ranges of numerical values.

In some embodiments, the term “substantially all” means at least about 90%, 95%, 96%, 97%, 98% or 99%. In some embodiments, the term “substantially free” means no more than about 10%, 5%, 4%, 3%, 2% or 1% by weight or molarity, or no more than about 1000 ppm, 500 ppm, 400 ppm, 300 ppm, 200 ppm or 100 ppm.

In some embodiments, the term “substantially pure” means at least about 90%, 95%, 96%, 97%, 98% or 99% pure. In some embodiments, the term “substantially stereochemically pure” means at least about 80% enantiomeric excess (90% of one enantiomer—10% of the other enantiomer) or 90% enantiomeric excess (95% of one enantiomer—5% of the other enantiomer), or at least about 80% diastereomeric excess (90% of one diastereomer—10% of all the other diastereomers) or at least about 90% diastereomeric excess (95% of one diastereomer—5% of all the other diastereomers).

Whenever the term “at least” or “greater than” precedes the first numerical value in a series of two or more numerical values, the term “at least” or “greater than” applies to each one of the numerical values in that series of numerical values.

Whenever the term “no more than” or “less than” precedes the first numerical value in a series of two or more numerical values, the term “no more than” or “less than” applies to each one of the numerical values in that series of numerical values.

The term “pharmaceutically acceptable” means that a substance (e.g., an active ingredient or an excipient) is generally safe, non-toxic and suitable for use in contact with the cells, tissues and organs of a subject without excessive irritation, allergic response, immunogenicity and other adverse reaction. A “pharmaceutically acceptable” excipient or carrier of a pharmaceutical composition is also compatible with the other ingredients of the composition.

The term “therapeutically effective amount” refers to an amount of a compound that, when administered to a subject or used ex vivo, is sufficient to prevent, reduce the risk of developing, delay the onset of, slow the progression of or cause regression of the medical condition being treated, or to alleviate or ameliorate to some extent the medical condition or one or more symptoms or complications of that condition, at least in some fraction of the subjects taking that compound or undergoing ex vivo treatment with that compound. The term “therapeutically effective amount” also refers to an amount of a compound that is sufficient to elicit the biological or medical response of a cell, tissue, organ, system, animal or human which is sought by a researcher, veterinarian, medical doctor or clinician.

The terms “treat,” “treating” and “treatment” include alleviating, ameliorating, reducing the incidence, frequency or severity of, slowing or stopping the progress of, reversing or abrogating a medical condition or one or more symptoms or complications associated with the condition, and alleviating, ameliorating or eradicating one or more causes of the condition. Reference to “treatment” of a medical condition includes prevention of the condition. The terms “prevent,” “preventing” and “prevention” include precluding, reducing the risk or likelihood of developing, and delaying the onset of a medical condition or one or more symptoms or complications associated with the condition.

The term “medical conditions” (or “conditions” for brevity) includes diseases and disorders. The terms “diseases” and “disorders” are used interchangeably herein.

The term “subject” refers to an animal, including but not limited to a mammal, such as a primate (e.g., a human, a chimpanzee or a monkey), a rodent (e.g., a rat, a mouse, a guinea pig, a gerbil or a hamster), a lagomorph (e.g., a rabbit), a bovine (e.g., a cattle), a suid (e.g., a pig), a caprine (e.g., a sheep), an equine (e.g., a horse), a canine (e.g., a dog) or a feline (e.g., a cat). The terms “subject” and “patient” may be used interchangeably herein in reference to a subject/patient (e.g., a mammalian subject/patient such as a human subject/patient) having a medical condition.

A “modulator” of, e.g., a receptor or enzyme can be an activator or inhibitor of that receptor or enzyme, and can increase or reduce the activity or/and the level of that receptor or enzyme.

The term “compound” or the like (e.g., “molecule”) encompasses salts, solvates, hydrates, clathrates and polymorphs of that compound or a salt of that compound. A “solvate” of a compound comprises a stoichiometric or non-stoichiometric amount of a solvent molecule (e.g., water, acetone or an alcohol [e.g., ethanol]) bound non-covalently to the compound. A “hydrate” of a compound comprises a stoichiometric or non-stoichiometric amount of water molecule bound non-covalently to the compound. A “clathrate” of a compound contains molecules of a substance (e.g., a solvent) enclosed in a crystal structure of the compound. A “polymorph” of a compound is a particular crystalline form of the compound. The specific recitation of “salt,” “solvate,” “hydrate,” “clathrate” or “polymorph” with respect to a compound or a group of compounds in certain instances of the disclosure shall not be interpreted as an intended omission of any of these forms in other instances of the disclosure where the term “compound” or the like (e.g., “molecule”) is used, or where the compound or the group of compounds is mentioned or shown, without recitation of any of these forms, unless stated otherwise or the context clearly indicates otherwise.

Where a molecular weight is provided and not an absolute value, for example, of a polymer, then the molecular weight should be understood to be an average molecule weight, unless otherwise stated or understood from the context.

It should be understood that the order of steps or order for performing certain actions is immaterial so long as the present invention remain operable. Moreover, two or more steps or actions may be conducted simultaneously.

### Unnatural Peptide Assemblies

As disclosed herein, the present disclosure is directed to unnatural peptide assemblies of Formula I, or tautomers, pharmaceutically acceptable salts, solvates, hydrates, clathrates or polymorphs thereof,

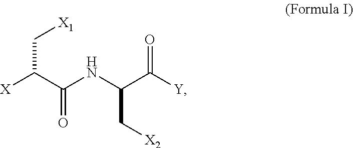

wherein X is selected from NH₂, NHX₃, OH,

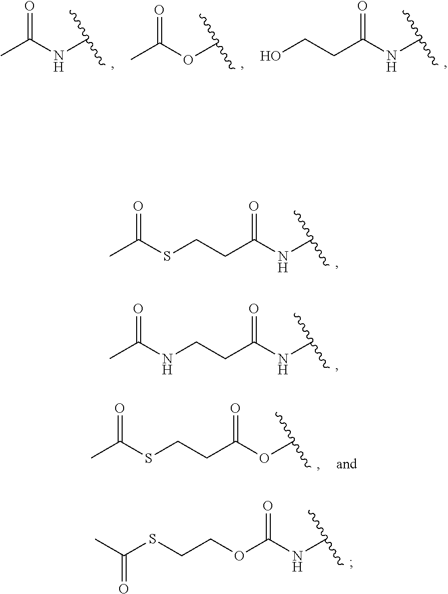

wherein X₁ is selected from biphenyl and phenyl; wherein X₂ is selected from biphenyl and phenyl; and wherein X₃ is selected from Glycine, D-Alanine, D-Serine, D-Lysine,

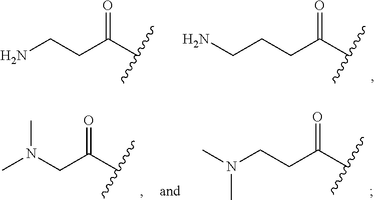

and wherein Y is selected from

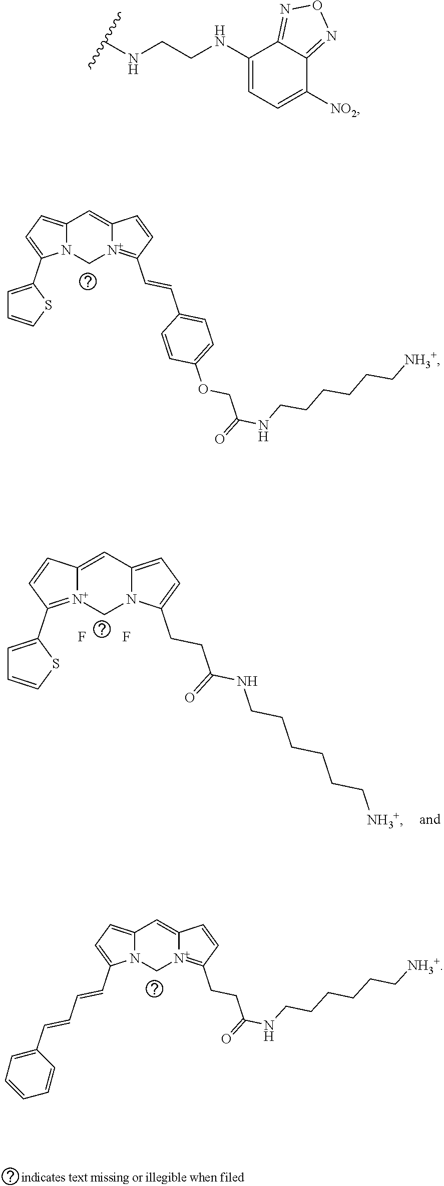

In some embodiments, X is NH₂, X₁ is biphenyl, X₂ is biphenyl, and Y is

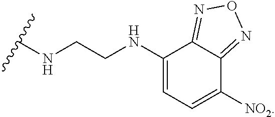 [SMILES: *NCCNc1ccc([N+](=O)[O-])c2nonc12]

In some embodiments, X is

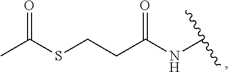 [SMILES: *NC(=O)CCSC(C)=O]

X₁ is biphenyl, X₂ is biphenyl, and Y is

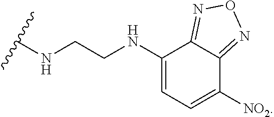 [SMILES: *NCCNc1ccc([N+](=O)[O-])c2nonc12]

In some embodiments, X is NHX₃ and X₃ is Glycine; X₁ is biphenyl and X₂ is biphenyl; and Y is

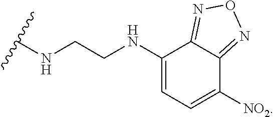 [SMILES: *NCCNc1ccc([N+](=O)[O-])c2nonc12]

In some embodiments, the unnatural peptide assembly contains an unnatural lipophilic motif, D-biphenylalanine-D-biphenylalanine (bb) and a fluorophore.

In some embodiments, the unnatural peptide assembly contains an unnatural lipophilic motif, D-biphenylalanine-D-biphenylalanine (bb), for self-assembly, and a fluorophore for imaging.

In some embodiments, the fluorophore is nitrobenzoxadiazole (NBD).

In some embodiments, the unnatural peptide assembly contains both D-biphenylalanine-D-biphenylalanine (bb) and nitrobenzoxadiazole (NBD) to form bb-NBD.

In some embodiments, the unnatural peptide assembly contains

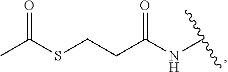 [SMILES: *NC(=O)CCSC(C)=O]

D-biphenylalanine-D-biphenylalanine (bb) and nitrobenzoxadiazole (NBD) to form AcS-bb-NBD.

In some embodiments, the unnatural peptide assembly contains Glycine, D-biphenylalanine-D-biphenylalanine (bb) and nitrobenzoxadiazole (NBD) to form Gbb-NBD.

In some embodiments, the unnatural peptide assembly is of Formula bb-NBD.

Formula bb-NBD is Formula II:

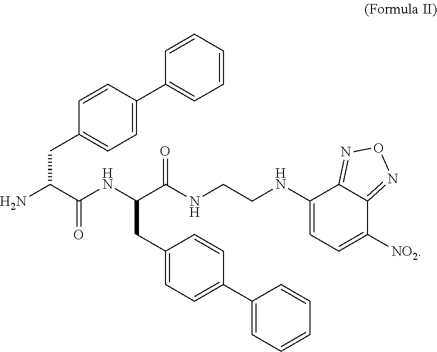 [SMILES: N[C@H](Cc1ccc(-c2ccccc2)cc1)C(=O)N[C@H](Cc1ccc(-c2ccccc2)cc1)C(=O)NCCNc1ccc([N+](=O)[O-])c2nonc12]

In some embodiments, the unnatural peptide assembly is of Formula AcS-bb-NBD.

Formula AcS-bb-NBD is Formula III:

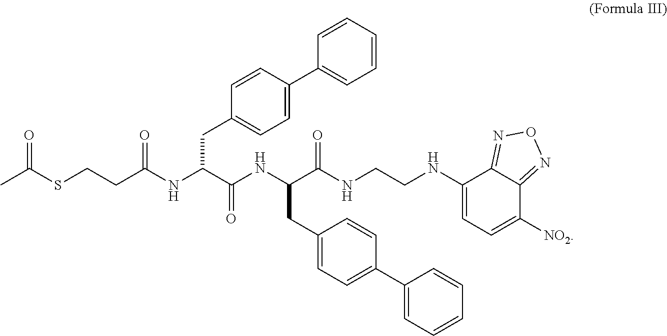 [SMILES: CC(=O)SCCC(=O)N[C@H](Cc1ccc(-c2ccccc2)cc1)C(=O)N[C@H](Cc1ccc(-c2ccccc2)cc1)C(=O)NCCNc1ccc([N+](=O)[O-])c2nonc12]

In some embodiments, the unnatural peptide assembly is of Formula Gbb-NBD.

### Formula Gbb-NBD is Formula IV:

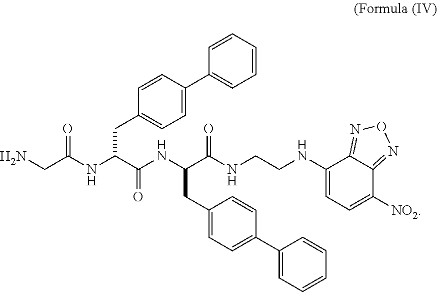 [SMILES: NCC(=O)N[C@H](Cc1ccc(-c2ccccc2)cc1)C(=O)N[C@H](Cc1ccc(-c2ccccc2)cc1)C(=O)NCCNc1ccc([N+](=O)[O-])c2nonc12]

As illustrated in the Examples that follow, the unnatural polypeptides of the present disclosure have been shown to modulate membrane biophysics for therapeutic purposes, and can manipulate the membrane biophysics of cancer cells by rapidly depleting cholesterol.

### Pharmaceutical Compositions

In some embodiments, the unnatural peptide assembly of Formula I, or a tautomer, a pharmaceutically acceptable salt, a solvate, a hydrate, a clathrate or a polymorph thereof.

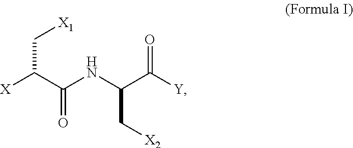

wherein X is selected from NH₂, NHX₃, OH,

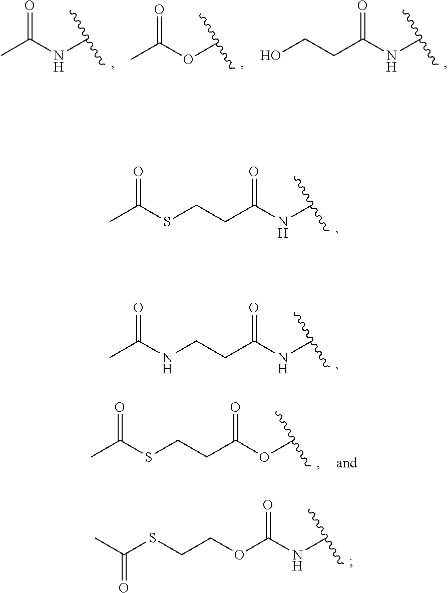

wherein X₁ is selected from biphenyl and phenyl; wherein X₂ is selected from biphenyl and phenyl; and wherein X₃ is selected from Glycine, D-Alanine, D-Serine, D-Lysine,

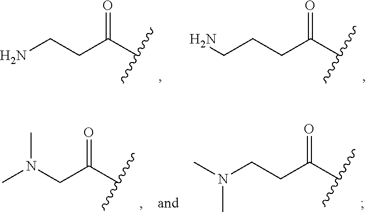

wherein Y is selected from

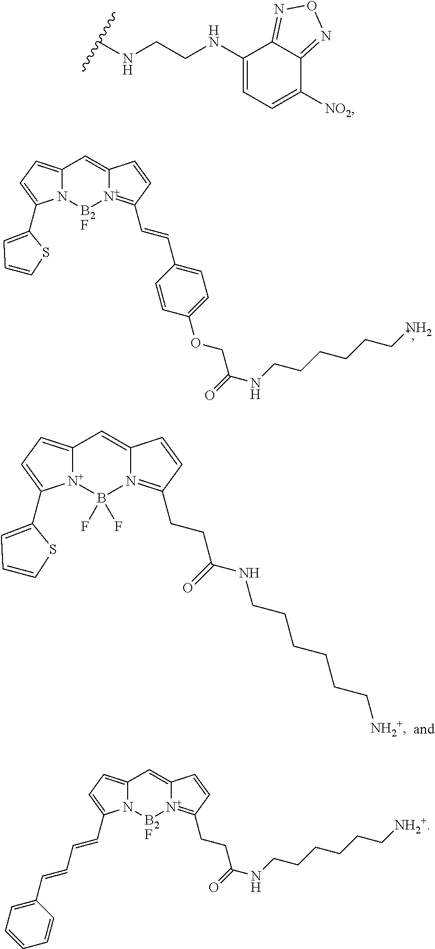

In some embodiments, the X is NH₂, X₁ is biphenyl, X₂ is biphenyl, and Y is

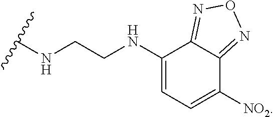 [SMILES: *NCCNc1ccc([N+](=O)[O-])c2nonc12]

In some embodiments, the X is

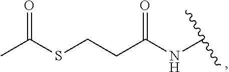 [SMILES: *NC(=O)CCSC(C)=O]

X₁ is biphenyl, X₂ is biphenyl, and Y is

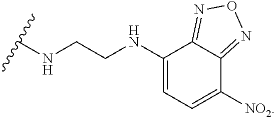 [SMILES: *NCCNc1ccc([N+](=O)[O-])c2nonc12]

In some embodiments, the X is NHX₃ and X₃ is Glycine; X₁ is biphenyl and X₂ is biphenyl; and Y is

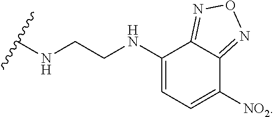 [SMILES: *NCCNc1ccc([N+](=O)[O-])c2nonc12]

In some embodiments, an unnatural peptide assembly contains an unnatural lipophilic motif, D-biphenylalanine-D-biphenylalanine (bb) and a fluorophore. In some embodiments, the fluorophore is nitrobenzoxadiazole (NBD).

In some embodiments, an unnatural peptide assembly contains an unnatural lipophilic motif, D-biphenylalanine-D-biphenylalanine (bb), for self-assembly, and a fluorophore for imaging.

In some embodiments, the unnatural peptide assembly is of Formula bb-NBD.

Formula bb-NBD is

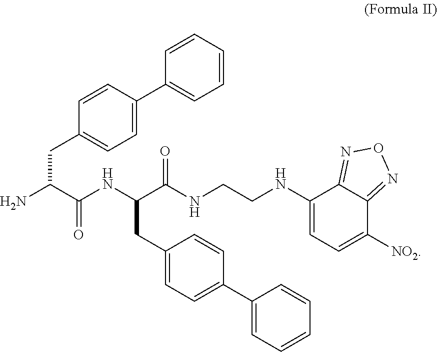 [SMILES: N[C@H](Cc1ccc(-c2ccccc2)cc1)C(=O)N[C@H](Cc1ccc(-c2ccccc2)cc1)C(=O)NCCNc1ccc([N+](=O)[O-])c2nonc12]

In some embodiments, the unnatural peptide assembly is of Formula AcS-bb-NBD.

Formula AcS-bb-NBD is

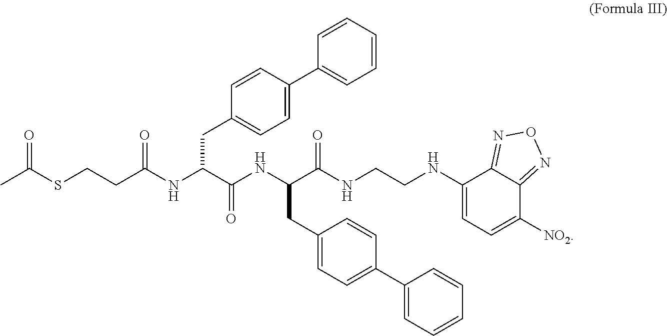 [SMILES: CC(=O)SCCC(=O)N[C@H](Cc1ccc(-c2ccccc2)cc1)C(=O)N[C@H](Cc1ccc(-c2ccccc2)cc1)C(=O)NCCNc1ccc([N+](=O)[O-])c2nonc12]

In some embodiments, the unnatural peptide assembly is of Formula Gbb-NBD.

Formula Gbb-NBD is

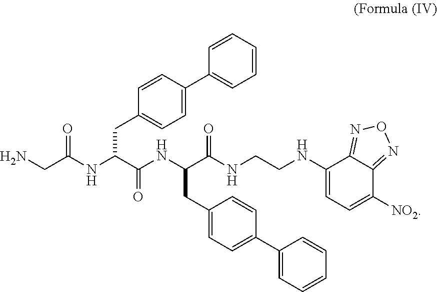 [SMILES: NCC(=O)N[C@H](Cc1ccc(-c2ccccc2)cc1)C(=O)N[C@H](Cc1ccc(-c2ccccc2)cc1)C(=O)NCCNc1ccc([N+](=O)[O-])c2nonc12]

In some embodiments, a pharmaceutical composition comprises an unnatural peptide assembly as taught and disclosed herein, or a pharmaceutically acceptable salt, a solvate, a hydrate, a clathrate or a polymorph thereof; and one or more pharmaceutically acceptable excipients or carriers. In some embodiments, the unnatural peptide assembly is in an aqueous medium.

In some embodiments, the pharmaceutical composition further comprises an additional therapeutic agent, wherein the additional therapeutic agent comprises alkylating agents, cytotoxic antibiotics, anti-metabolites, anti-microtubule agents, histone deacetylase inhibitors, kinase inhibitors, topoisomerase I inhibitors, topoisomerase II-targeting agents, DNA or RNA synthesis inhibitors, protein synthesis inhibitors, cell growth and differentiation regulators, cell proliferation inhibitors, apoptosis inducers, agonists/activators of tumor necrosis factor receptor superfamily member 4, agonists/activators of TNFRSF member 5, agonists/activators of TNFRSF member 9, agonists/activators of TNFRSF member 18, agonists/activators of toll-like receptors, cytokines and hormones, inhibitors of programmed cell death 1 (PD-1) receptor, inhibitors of cytotoxic T lymphocyte-associated protein 4, inhibitors of killer cell immunoglobulin-like receptors, and inhibitors of lymphocyte activation gene 3 (LAG-3) receptor.

Additional embodiments of the disclosure relate to pharmaceutical compositions comprising the unnatural peptide assemblies taught and described herein, or a tautomer, a pharmaceutically acceptable salt, a solvate, a hydrate, a clathrate or a polymorph thereof; and one or more pharmaceutically acceptable excipients or carriers. The compositions can optionally contain an additional therapeutic agent. A pharmaceutical composition contains a therapeutically effective amount, or any appropriate fraction thereof, of an unnatural peptide assembly; one or more pharmaceutically acceptable excipients or carriers; and optionally, a therapeutically effective amount of an additional therapeutic agent The pharmaceutical composition can be formulated for administration to a subject in need thereof for therapeutic use. For purposes of the content of a pharmaceutical composition, the term “active ingredient,” “active agent,” “therapeutic agent,” “drug” or the like encompasses a prodrug.

A pharmaceutical composition can contain the unnatural peptide assembly; and optionally, an additional therapeutic agent in substantially pure form. In some embodiments, the purity of the unnatural peptide assembly and the optional additional therapeutic agent independently is at least about 95%, 96%, 97%, 98% or 99%. In addition, a pharmaceutical composition is substantially free of contaminants or impurities. In some embodiments, the level of contaminants or impurities other than residual solvent in a pharmaceutical composition is no more than about 5%, 4%, 3%, 2% or 1% relative to the combined weight of the intended active and inactive ingredients. Pharmaceutical compositions generally are prepared according to current good manufacturing practice (GMP), as recommended or required by, e.g., the Federal Food, Drug, and Cosmetic Act § 501(a)(2)(B) and the International Conference on Harmonisation Q7 Guideline.

Pharmaceutical compositions/formulations can be prepared in sterile form. For example, pharmaceutical compositions/formulations for parenteral administration by injection or infusion generally are sterile. Sterile pharmaceutical compositions/formulations are compounded or manufactured according to pharmaceutical-grade sterilization standards known to those of skill in the art, such as those disclosed in or required by the United States Pharmacopeia Chapters 797, 1072 and 1211, and 21 Code of Federal Regulations 211.

Pharmaceutically acceptable excipients and carriers include pharmaceutically acceptable substances, materials and vehicles. Non-limiting examples of types of excipients include liquid and solid fillers, diluents, binders, lubricants, glidants, surfactants, dispersing agents, disintegration agents, emulsifying agents, wetting agents, suspending agents, thickeners, solvents, isotonic agents, buffers, pH adjusters, absorption-delaying agents, stabilizers, antioxidants, preservatives, antimicrobial agents, antibacterial agents, antifungal agents, chelating agents, adjuvants, sweetening agents, flavoring agents, coloring agents, encapsulating materials and coating materials. The use of such excipients in pharmaceutical formulations is known in the art. For example, conventional vehicles and carriers include without limitation oils (e.g., vegetable oils such as olive oil and sesame oil), aqueous solvents {e.g., saline, buffered saline (e.g., phosphate-buffered saline [PBS]) and isotonic solutions (e.g., Ringer's solution)}, and organic solvents (e.g., dimethyl sulfoxide [DMSO] and alcohols [e.g., ethanol, glycerol and propylene glycol]). Except insofar as any conventional excipient or carrier is incompatible with the active ingredient, the disclosure encompasses the use of conventional excipients and carriers in formulations containing unnatural peptide assemblies. See, e.g., Remington: The Science and Practice of Pharmacy, 21st Ed., Lippincott Williams & Wilkins (Philadelphia, Pennsylvania) (2005); Handbook of Pharmaceutical Excipients, 5th Ed., Rowe et al., Eds., The Pharmaceutical Press and the American Pharmaceutical Association (2005); Handbook of Pharmaceutical Additives, 3rd Ed., Ash and Ash, Eds., Gower Publishing Co. (2007); and Pharmaceutical Pre-formulation and Formulation, Gibson, Ed., CRC Press (Boca Raton, Florida) (2004).

Appropriate formulation can depend on various factors, such as the route of administration chosen. Potential routes of administration of pharmaceutical compositions comprising unnatural peptide assemblies include without limitation oral, parenteral (including intradermal, subcutaneous, intramuscular, intravascular, intravenous, intraarterial, intraperitoneal, intracavitary, intramedullary, intrathecal and topical), and topical (including dermal/epicutaneous, transdermal, mucosal, transmucosal, intranasal [e.g., by nasal spray or drop], ocular/intraocular [e.g., by eye drop], pulmonary [e.g., by oral or nasal inhalation], buccal, sublingual, rectal [e.g., by suppository], and vaginal [e.g., by suppository]). Topical formulations can be designed to produce a local or systemic therapeutic effect. In some embodiments, the unnatural peptide assemblies are administered as an oral dosage form such as a tablet, capsule or pill.

As an example, formulations of the unnatural peptide assemblies suitable for oral administration can be presented in discrete units adapted for instant, controlled or sustained release as, e.g., boluses; capsules (including push-fit capsules and soft capsules), tablets, pills, cachets or lozenges; as powders or granules; as semisolids, electuaries, pastes or gels; as solutions or suspensions in an aqueous liquid or/and a non-aqueous liquid; or as oil-in-water liquid emulsions or water-in-oil liquid emulsions.

Push-fit capsules or two-piece hard gelatin capsules can contain unnatural peptide assemblies in admixture with, e.g., a filler or inert solid diluent (e.g., calcium carbonate, calcium phosphate, kaolin or lactose), a binder (e.g., a starch), a glidant or lubricant (e.g., talc or magnesium stearate), and a disintegrant (e.g., crospovidone), and optionally a stabilizer or/and a preservative. For soft capsules or single-piece gelatin capsules, unnatural peptide assemblies can be dissolved or suspended in a suitable liquid (e.g., liquid polyethylene glycol or an oil medium, such as a fatty oil, peanut oil, olive oil or liquid paraffin), and the liquid-filled capsules can contain one or more other liquid excipients or/and semi-solid excipients, such as a stabilizer or/and an amphiphilic agent (e.g., a fatty acid ester of glycerol, propylene glycol or sorbitol).

Compositions for oral administration can also be formulated as solutions or suspensions in an aqueous liquid or/and a non-aqueous liquid, or as oil-in-water liquid emulsions or water-in-oil liquid emulsions. Dispersible powder or granules of unnatural peptide assemblies can be mixed with any suitable combination of an aqueous liquid, an organic solvent or/and an oil and any suitable excipients (e.g., any combination of a dispersing agent, a wetting agent, a suspending agent, an emulsifying agent or/and a preservative) to form a solution, suspension or emulsion.

The unnatural peptide assemblies can also be formulated for parenteral administration by injection or infusion to circumvent gastrointestinal absorption and first-pass metabolism. An exemplary parenteral route is intravenous. Additional advantages of intravenous administration include direct administration of a therapeutic agent into systemic circulation to achieve a rapid systemic effect, and the ability to administer the agent continuously or/and in a large volume if desired. Formulations for injection or infusion can be in the form of, e.g., solutions, suspensions or emulsions in oily or aqueous vehicles, and can contain excipients such as suspending agents, dispersing agents or/and stabilizing agents. For example, aqueous or non-aqueous (e.g., oily) sterile injection solutions can contain unnatural peptide assemblies along with excipients such as an antioxidant, a buffer, a bacteriostat and solutes that render the formulation isotonic with the blood of the subject. Aqueous or non-aqueous sterile suspensions can contain unnatural peptide assemblies along with excipients such as a suspending agent and a thickening agent, and optionally a stabilizer and an agent that increases the solubility of the unnatural peptide assemblies to allow for the preparation of a more concentrated solution or suspension. As another example, a sterile aqueous solution for injection or infusion (e.g., subcutaneously or intravenously) can contain unnatural peptide assemblies, an isotonic agent (e.g., sodium chloride), a buffering agent (e.g., sodium citrate), a preservative (e.g., meta-cresol), and optionally a base (e.g., NaOH) or/and an acid (e.g., HCl) to adjust pH.

Topical formulations for application to the skin or mucosa can be useful for transdermal or transmucosal administration of a therapeutic agent to the local target site of action, or into the blood for systemic distribution. Advantages of topical administration can include circumvention of the GI tract (including enzymes and acid in the GI tract and absorption through it) and first-pass metabolism; delivery of a therapeutic agent with a short half-life, a small therapeutic index or/and low oral bioavailability; controlled, continuous and sustained release of the therapeutic agent; a more uniform plasma level or delivery profile of the therapeutic agent; lower dose and less frequent dosing of the therapeutic agent; reduction of systemic side effects (e.g., side effects caused by a temporary overdose or an overly high peak plasma drug concentration); minimal or no invasiveness; ease of self-administration; and increased patient compliance.

Compositions suitable for topical administration include without limitation liquid or semi-liquid preparations such as sprays, gels, liniments, lotions, oil-in-water or water-in-oil emulsions such as creams, foams, ointments and pastes, and solutions or suspensions such as drops (e.g., eye drops, nose drops and ear drops). See Remington: The Science and Practice of Pharmacy, 21st Ed., Lippincott Williams & Wilkins (Philadelphia, Pennsylvania [2005]). Various excipients can be included in a topical formulation. For example, solvents, including a suitable amount of an alcohol, can be used to solubilize the active agent. Other optional excipients include without limitation gelling agents, thickening agents, emulsifiers, surfactants, stabilizers, buffers, antioxidants, preservatives, cooling agents (e.g. menthol), opacifiers, fragrances and colorants. For an active agent having a low rate of permeation through the skin or mucosal tissue, a topical formulation can contain a chemical permeation enhancer (e.g., a fatty acid ester [e.g., isopropyl myristate or isopropyl palmitate], a fatty acid [e.g., palmitic acid, oleic acid or palmitoleic acid], or/and an alcohol [e.g., propylene glycol or a fatty alcohol such as geraniol or farnesol]) to increase the permeation of the active agent through the skin or mucosal tissue. A topical formulation can also contain an irritation-mitigating excipient that reduces any irritation to the skin or mucosa caused by the active agent, the chemical permeation enhancer or any other component of the formulation. In some embodiments, a topical composition comprises a therapeutic agent dissolved, dispersed or suspended in a carrier. The carrier can be in the form of, e.g., a solution, a suspension, an emulsion, an ointment or a gel base, and can contain, e.g., petrolatum, lanolin, a wax (e.g., bee wax), mineral oil, a long-chain alcohol, polyethylene glycol or polypropylene glycol, a diluent (e.g., water or/and an alcohol [e.g., ethanol or propylene glycol]), a gel, an emulsifier, a thickening agent, a stabilizer or a preservative, or any combination thereof. A topical formulation can be administered by means of, e.g., a transdermal or transmucosal delivery device, such as a transdermal patch, a microneedle patch or an iontophoresis device. A topical composition can deliver a drug transdermally or transmucosally via a concentration gradient (with or without the use of a chemical permeation enhancer) or an active mechanism (e.g., iontophoresis or microneedles).

For topical administration, the unnatural peptide assemblies can be formulated as, e.g., a buccal or sublingual tablet or pill. Advantages of a buccal or sublingual tablet or pill include avoidance of gastrointestinal absorption and first-pass metabolism, and rapid absorption into systemic circulation. A buccal or sublingual tablet or pill can be designed to provide faster release of the unnatural peptide assemblies for more rapid uptake of it into systemic circulation. In addition to a therapeutically effective amount of unnatural peptide assemblies, the buccal or sublingual tablet or pill can contain suitable excipients, including without limitation any combination of fillers and diluents (e.g., mannitol and sorbitol), binding agents (e.g., sodium carbonate), wetting agents (e.g., sodium carbonate), disintegrants (e.g., crospovidone and croscarmellose sodium), lubricants (e.g., silicon dioxide [including colloidal silicon dioxide] and sodium stearyl fumarate), stabilizers (e.g., sodium bicarbonate), flavoring agents (e.g., spearmint flavor), sweetening agents (e.g., sucralose), and coloring agents (e.g., yellow iron oxide).

For topical administration, the unnatural peptide assemblies can also be formulated for intranasal administration. The nasal mucosa provides a big surface area, a porous endothelium, a highly vascular subepithelial layer and a high absorption rate, and hence allows for high bioavailability. Moreover, intranasal administration avoids first-pass metabolism and can introduce a significant concentration of the unnatural peptide assemblies to the central nervous system (CNS). An intranasal formulation can comprise unnatural peptide assemblies along with excipients, such as a solubility enhancer (e.g., propylene glycol), a humectant (e.g., mannitol or sorbitol), a buffer and water, and optionally a preservative (e.g., benzalkonium chloride), a mucoadhesive agent (e.g., hydroxyethylcellulose) or/and a penetration enhancer. An intranasal solution or suspension formulation can be administered to the nasal cavity by any suitable means, including but not limited to a dropper, a pipette, or a spray using, e.g., a metering atomizing spray pump.

An additional mode of topical administration of unnatural peptide assemblies is pulmonary, including by oral inhalation and nasal inhalation. A pulmonarily administered drug can treat a lung disease or/and a systemic disease, as the lungs serve as a portal to the systemic circulation. Advantages of pulmonary drug delivery include, for example: 1) avoidance of first-pass metabolism; 2) fast drug action; 3) large surface area of the alveolar region for absorption, high permeability of the lungs (thin air-blood barrier), and profuse vasculature of the airways; 4) reduced extracellular enzyme levels compared to the GI tract due to the large alveolar surface area; and 5) smaller doses to achieve equivalent therapeutic effect compared to other oral routes, and hence reduced systemic side effects. Oral inhalation can also enable more rapid action of a drug in the CNS. An advantage of oral inhalation over nasal inhalation includes deeper penetration/deposition of the drug into the lungs, although nasal inhalation can deliver the drug into systemic circulation transmucosally in the nasal cavity as well as in the lungs. Oral or nasal inhalation can be achieved by means of, e.g., a metered-dose inhaler (MDI), a dry powder inhaler (DPI) or a nebulizer, as is known in the art. In certain embodiments, a sterile aqueous solution for oral inhalation contains unnatural peptide assemblies, sodium chloride, a buffering agent (e.g., sodium citrate), optionally a preservative (e.g., meta-cresol), and optionally a base (e.g., NaOH) or/and an acid (e.g., HCl) to adjust pH.

In some embodiments, the unnatural peptide assemblies are administered transdermally. In some embodiments, the topical composition or the transdermal delivery system comprises a chemical permeation enhancer (e.g., a surfactant [e.g., sodium laureth sulfate], optionally in combination with an aromatic compound [e.g., phenylpiperazine]) that facilitates the transport of the unnatural peptide assemblies across the skin. In further embodiments, the unnatural peptide assemblies are administered via a transdermal patch. In some embodiments, the transdermal patch is a reservoir-type patch comprising an impermeable backing layer/film, a liquid- or gel-based drug reservoir, a semi-permeable membrane that serves as a rate-limiting or rate-controlling diffusion barrier, and a skin-contacting adhesive layer. The semi-permeable membrane can be composed of, e.g., a suitable polymeric material such as cellulose nitrate or acetate, polyisobutene, polypropylene, polyvinyl acetate or a polycarbonate. In other embodiments, the transdermal patch is a drug-in-adhesive patch comprising an impermeable backing layer/film and a skin-contacting adhesive layer incorporating the drug in a polymeric or viscous adhesive. The adhesive of the drug-loaded, skin-contacting adhesive layer can be, e.g., a pressure-sensitive adhesive (PSA), such as a PSA composed of an acrylic polymer (e.g., polyacrylate), a polyalkylene (e.g., polyisobutylene) or a silicone-based polymer (e.g., silicone-2675 or silicone-2920). Transdermal drug-delivery systems, including patches, can be designed to provide controlled and prolonged release of a drug over a period of about 1 week, 2 weeks, 3 weeks, 1 month or longer.

In some embodiments, the unnatural peptide assemblies are delivered from a sustained-release composition. As used herein, the term “sustained-release composition” encompasses sustained-release, prolonged-release, extended-release, delayed-release, slow-release and controlled-release compositions, systems and devices. Advantages of a sustained-release composition include without limitation a more uniform blood level of the drug (e.g., avoidance of wide peak-to-trough fluctuations), delivery of a therapeutically effective amount of the drug over a prolonged time period, reduced frequency of administration, and reduced side effects (e.g., avoidance of a drug overdose). In some embodiments, the sustained-release composition delivers the unnatural peptide assemblies over a period of at least about 1 day, 2 days, 3 days, 1 week, 2 weeks, 3 weeks, 1 month, 2 months, 3 months or longer.

In some embodiments, the sustained-release composition is a drug-encapsulation system, such as nanoparticles, microparticles or a capsule made of, e.g., a biodegradable polymer or/and a hydrogel. In certain embodiments, the sustained-release composition comprises a hydrogel. Non-limiting examples of polymers of which a hydrogel can be composed include polyvinyl alcohol, acrylate polymers (e.g., sodium polyacrylate), and other homopolymers and copolymers having a relatively large number of hydrophilic groups (e.g., hydroxyl or/and carboxylate groups). In other embodiments, the sustained-release drug-encapsulation system comprises a membrane-enclosed reservoir, wherein the reservoir contains a drug and the membrane is permeable to the drug. Such a drug-delivery system can be in the form of, e.g., a transdermal patch.

In some embodiments, the sustained-release composition is formulated as polymeric nanoparticles or microparticles, wherein the polymeric particles can be delivered, e.g., by injection or from an implant. In some embodiments, the polymeric implant or polymeric nanoparticles or microparticles are composed of a biodegradable polymer. In certain embodiments, the biodegradable polymer comprises lactic acid or/and glycolic acid [e.g., an L-lactic acid-based copolymer, such as poly(L-lactide-co-glycolide) or poly(L-lactic acid-co-D,L-2-hydroxyoctanoic acid)]. For example, biodegradable polymeric microspheres composed of polylactic acid or/and polyglycolic acid can serve as sustained-release pulmonary drug-delivery systems. The biodegradable polymer of the polymeric implant or polymeric nanoparticles or microparticles can be selected so that the polymer substantially completely degrades around the time the period of treatment is expected to end, and so that the byproducts of the polymer's degradation, like the polymer, are biocompatible.

In further embodiments, the sustained-release composition comprises a dendrimer. In certain embodiments, the dendrimer is a water-soluble dendrimer, such as a poly(amidoamine) (PAMAM) dendrimer. In some embodiments, a dendrimer encapsulates a drug through the formation of a dendrimer-drug supramolecular assembly. In other embodiments, the sustained-release composition comprises a water-soluble polymer [e.g., poly(DL-lactide)] or a liposome encapsulating a drug complexed with a dendrimer.

In other embodiments, the sustained-release composition is an oral dosage form, such as a tablet or capsule. For example, a drug can be embedded in an insoluble porous matrix such that the dissolving drug must make its way out of the matrix before it can be absorbed through the GI tract. Alternatively, a drug can be embedded in a matrix that swells to form a gel through which the drug exits. Sustained release can also be achieved by way of a single-layer or multi-layer osmotic controlled-release oral delivery system (OROS). An OROS is a tablet with a semi-permeable outer membrane and one or more small laser-drilled holes in it. As the tablet passes through the body, water is absorbed through the semi-permeable membrane via osmosis, and the resulting osmotic pressure pushes the drug out through the hole(s) in the tablet and into the GI tract where it can be absorbed.

For a delayed or sustained release of unnatural peptide assemblies, an unnatural peptide assembly can also be formulated as, e.g., a depot that can be implanted in or injected into a subject, e.g., intramuscularly, intracutaneously or subcutaneously. A depot formulation can be designed to deliver unnatural peptide assemblies over an extended period of time, e.g., over a period of at least about 1 week, 2 weeks, 3 weeks, 1 month, 6 weeks, 2 months, 3 months or longer. For example, unnatural peptide assemblies can be formulated with a polymeric material (e.g., polyethylene glycol [PEG], polylactic acid [PLA] or polyglycolic acid [PGA], or a copolymer thereof [e.g., PLGA or PLA-PEG]), with a hydrophobic material (e.g., as an emulsion in an oil) and/or an ion-exchange resin, as a more lipophilic derivative (e.g., as an ester of or a salt with a fatty acid such as a C₈-C₂₀ fatty acid [e.g., decanoic acid]), or as a sparingly soluble derivative (e.g., a sparingly soluble salt). As an illustrative example, unnatural peptide assemblies can be incorporated or embedded in sustained release microparticles composed of PLGA and formulated as a monthly depot.

The unnatural peptide assemblies can also be contained or dispersed in a matrix material. The matrix material can comprise a polymer (e.g., ethylene-vinyl acetate) and controls the release of the drug by controlling dissolution and/or diffusion of the drug from, e.g., a reservoir, and can enhance the stability of the drug while contained in the reservoir. Such a release system can be designed as a sustained-release system, can be configured as, e.g., a transdermal or transmucosal patch, and can contain an excipient that can accelerate the drug's release, such as a water-swellable material (e.g., a hydrogel) that aids in expelling the drug out of the reservoir. U.S. Pat. Nos. 4,144,317 and 5,797,898 describe examples of such a release system.

The release system can provide a temporally modulated release profile (e.g., pulsatile release) when time variation in plasma levels is desired, or a more continuous or consistent release profile when a constant plasma level is desired. Pulsatile release can be achieved from an individual reservoir or from a plurality of reservoirs. For example, where each reservoir provides a single pulse, multiple pulses (“pulsatile” release) are achieved by temporally staggering the single pulse release from each of multiple reservoirs. Alternatively, multiple pulses can be achieved from a single reservoir by incorporating several layers of a release system and other materials into a single reservoir. Continuous release can be achieved by incorporating a release system that degrades, dissolves, or allows diffusion of a drug through it over an extended time period. In addition, continuous release can be approximated by releasing several pulses of a drug in rapid succession (“digital” release). An active release system can be used alone or in conjunction with a passive release system, as described in U.S. Pat. No. 5,797,898.

In addition, pharmaceutical compositions comprising unnatural peptide assemblies can be formulated as, e.g., liposomes, micelles (e.g., those composed of biodegradable natural or/and synthetic polymers, such as lactosomes), nanoparticles (e.g., lipid nanoparticles such as solid lipid nanoparticles), microparticles or microspheres, whether or not designed for sustained release. For example, liposomes can be used as sustained-release pulmonary drug-delivery systems that deliver drugs to the alveolar surface for treatment of lung diseases and systemic diseases. As another example, lipid nanoparticles containing a lipophilic drug can be delivered into the lungs by oral inhalation for treatment of a lung disorder or a systemic disorder.

In some embodiments, liposomes or micelles are composed of one or more phospholipids. Phospholipids include without limitation phosphatidic acids (e.g., DEPA, DLPA, DMPA, DOPA, DPPA and DSPA), phosphatidylcholines (e.g., DDPC, DEPC, DLPC, DLOPC, DMPC, DOPC, DPPC, DSPC, MPPC, MSPC, PLPC, PMPC, POPC, PSPC, SMPC, SOPC and SPPC), phosphatidylethanolamines (e.g., DEPE, DLPE, DMPE, DOPE, DPPE, DSPE and POPE), phosphatidylglycerols (e.g., DEPG, DLPG, DMPG, DOPG, DPPG, DSPG and POPG), phosphatidylserines (e.g., DLPS, DMPS, DOPS, DPPS and DSPS), and salts (e.g., sodium and ammonium salts) thereof. In certain embodiments, liposomes or micelles are composed of one or more phosphatidylcholines. Liposomes have a hydrophilic core, so liposomes are particularly suited for delivery of more hydrophilic drugs, whereas micelles have a hydrophobic core, so micelles are particularly suited for delivery of more hydrophobic drugs. Liposomes and micelles can permeate across biological membranes. Moreover, liposomes and micelles composed of a fusogenic lipid (e.g., DPPG) can fuse with the plasma membrane of cells and thereby deliver a drug into those cells. Liposomes and micelles can provide sustained release of a drug based in part on the rate of degradation of the liposomes and micelles.

The pharmaceutical compositions can be manufactured in any suitable manner known in the art, such as by means of conventional mixing, dissolving, suspending, granulating, dragee-making, levigating, emulsifying, encapsulating, entrapping or compressing processes, or any combination thereof.

The unnatural peptide assemblies can be presented in unit dosage form as a single dose wherein all active and inactive ingredients are combined in a suitable system, and components do not need to be mixed to form the unnatural peptide assemblies to be administered. A unit dosage form generally contains a therapeutically effective dose of the drug, but can contain an appropriate fraction thereof so that taking multiple unit dosage forms achieves the therapeutically effective dose. Examples of a unit dosage form include a tablet, capsule, or pill for oral uptake; a solution in a pre-filled syringe of a single-use pen or a pen with a dose counter for parenteral (e.g., intravenous, subcutaneous or intramuscular) injection; a capsule, cartridge or blister pre-loaded in or manually loaded into an inhaler; and a reservoir-type transdermal patch or a drug-in-adhesive patch.

Alternatively, the unnatural peptide assemblies can be presented as a kit in which the active ingredient, excipient(s) and carrier(s) [e.g., solvent(s)] are provided in two or more separate containers (e.g., ampules, vials, tubes, bottles or syringes) and need to be combined to form the composition to be administered. The kit can contain instructions for storing, preparing and administering the composition (e.g., a solution to be injected parenterally).

A kit can contain all active and inactive ingredients in unit dosage form or the active ingredient and inactive ingredients in two or more separate containers, and can contain instructions for administering or using the pharmaceutical composition to treat a medical condition. A kit can further contain a device for delivering the composition, such as a needle and a syringe, an injection pen, an inhaler or a transdermal patch.

In some embodiments, a kit contains unnatural peptide assemblies or a pharmaceutically acceptable salt, solvate, hydrate, clathrate or polymorph thereof, or a pharmaceutical composition comprising the same, and instructions for administering or using the unnatural peptide assemblies or the composition to treat a medical condition (e.g., a tumor or cancer, a metabolic disorder or a liver disorder). In some embodiments, the kit further contains a device for delivering the unnatural peptide assemblies or the composition, such as an injection pen, an inhaler or a transdermal patch.

Another aspect of the present disclosure relates to a pharmaceutical composition that includes the unnatural peptide assemblies, as described herein, in an aqueous medium.

In certain embodiments, the unnatural peptide assemblies in the pharmaceutical composition form micelle structures. Additionally, the pharmaceutical composition can further contain a therapeutic agent encapsulated within the micelle structures. These therapeutic agents can be hydrophobic.

### Therapeutic Uses of Unnatural Peptide Assemblies

Cholesterol is crucial for both membrane integrity and cancer development. Studies have shown its role in promoting colon, breast, and prostate cancers. This makes it an attractive target for therapy.

In some embodiments, the unnatural peptide assemblies disclosed herein are used to treat a tumor or cancer.

In some embodiments, the tumor or cancer is selected from solid tumors, head and neck tumors and cancers (e.g., oral squamous cell carcinoma, esophageal squamous cell carcinoma, nasopharyngeal carcinoma and papillary thyroid cancer), tumors and cancers of the CNS (e.g., glioblastoma, glioma and neuroblastoma), lung cancer (e.g., non-small cell lung cancer), gynecological cancers (e.g., breast cancer, ovarian cancer [e.g., high-grade serous carcinoma], and uterine cancer [e.g., cervical cancer and endometrial cancer]), cancers of the gastrointestinal tract (e.g., stomach cancer [e.g., gastric carcinoma] and colorectal cancer), liver cancer (e.g., hepatocellular carcinoma and intrahepatic cholangiocarcinoma), pancreatic cancer (e.g., pancreatic adenocarcinoma), urological cancers (e.g., kidney cancer [e.g., renal clear cell carcinoma and renal papillary cell carcinoma], bladder cancer and prostate cancer), skin cancer (e.g., melanoma), sarcomas, and hematological and lymphoid cancers (e.g., leukemias and lymphomas [e.g., non-Hodgkin lymphomas such as diffuse large B-cell lymphoma]). In some embodiments, the tumor or cancer is characterized by intra-abdominal, peritoneal or/and omental metastasis, such as ovarian cancer (e.g., ovarian carcinoma), endometrial cancer, gastrointestinal cancer (e.g., gastric cancer or colorectal cancer), or pancreatic cancer (e.g., pancreatic ductal adenocarcinoma).

The therapeutically effective amount and frequency of administration of the unnatural peptide assemblies disclosed herein may depend on various factors, including the medical condition being treated, the severity of the condition, the potency of the compound, the route of administration, the age, body weight, general health, gender and diet of the subject, and the response of the subject to the treatment, and can be determined by the treating physician.

In some embodiments, the unnatural peptide assemblies of the pharmaceutical composition are present at a concentration of from about 100 nM to about 500 mM. Furthermore, the therapeutic agent can be present in an amount from about 1 nM to about 10 mM. Single doses of the pharmaceutical composition may contain from 0.1 qg to 0.1 g of the peptide, preferably 4 qg to 0.04 g or 400 qg to 0.4 g; and any effective amount of the therapeutic agent. Typically, single doses of the therapeutic agent range from 1 qg/kg body weight to 1000 mg/kg body weight (although lesser or greater dosages are also contemplated).

The dosing frequency may depend on, e.g., the route of administration chosen. For example, dosing by pulmonary administration (e.g., by oral inhalation) may occur more frequently (e.g., 2, 3 or 4 times daily). To more quickly establish a therapeutic level of the unnatural peptide assemblies, a loading dose of the unnatural peptide assemblies that is greater (e.g., about 2- or 3-fold greater) than the maintenance dose can be administered at the beginning (e.g., in the first three days) of treatment followed by administration of the maintenance dose.

The unnatural peptide assemblies can be administered via any suitable route, which may depend on, e.g., the medical condition being treated and its location and the pharmacokinetics of the inhibitor. Potential routes of administration of include without limitation oral, parenteral (including intradermal, subcutaneous, intramuscular, intravascular, intravenous, intra-arterial, intraperitoneal, intracavitary, intramedullary, intrathecal and topical), and topical (including dermal/epicutaneous, transdermal, mucosal, transmucosal, intranasal [e.g., by nasal spray or drop], ocular/intraocular [e.g., by eye drop], pulmonary [e.g., by oral or nasal inhalation], buccal, sublingual, rectal [e.g., by suppository], and vaginal [e.g., by suppository]).

In some embodiments, the unnatural peptide assemblies disclosed herein are administered orally. In other embodiments, unnatural peptide assemblies are administered parenterally, such as intravenously, subcutaneously or intramuscularly. In further embodiments, unnatural peptide assemblies are administered intratumorally to treat a tumor or cancer.

The length of treatment with unnatural peptide assemblies can be determined by the treating physician to achieve the desired outcome. In some embodiments, unnatural peptide assemblies are administered for at least about 1 week, 2 weeks, 3 weeks or 4 weeks (1 month). In other embodiments, unnatural peptide assemblies are administered for at least about 6 weeks, 2 months, 3 months, 6 months, 1 year, 2 years, 3 years or longer.

In some embodiments, the unnatural peptide assemblies disclosed herein can inhibit proliferation or growth of cancer cells by contacting the cancer cells. In some embodiments, the contacting occurs in vivo, ex vivo or in vitro.

In some embodiments, the cancer cells are cervical cancer cells, prostate cancer cells, or liver cancer cells.

In some embodiments, the unnatural peptide assemblies disclosed herein can modulate membrane biophysics for therapeutic purposes.

In some embodiments, the unnatural peptide assemblies disclosed herein can manipulate the membrane biophysics of cancer cells by rapidly depleting cholesterol.

In some embodiments, the disclosed unnatural peptide assembly self-assembles into micelles and is rapidly taken up by cancer cells, especially when combined with an acute cholesterol-depleting agent (MβCD).

### Combination Therapies

The unnatural peptide assemblies disclosed herein can optionally be used in combination with one or more additional therapeutic agents to treat a medical condition. In some embodiments, unnatural peptide assemblies are used in combination with one or more additional antitumor/anticancer agents to treat a tumor or cancer.

Antitumor/anticancer agents include without limitation:
1) cytotoxic agents, including without limitation:
i) alkylating agents, such as aziridines (e.g., diaziquone, mytomycin and thiotepa), nitrogen mustards (e.g., mannomustine, mustine [mechlorethamine or chlormethine], aniline mustard, bendamustine, benzoic acid mustard, chlorambucil, C6-galactose mustard, melphalan, ossichlorin [nitromin], prednimustine, uramustine, nitrogen mustard carbamates [e.g., estramustine], and oxazaphosphorines [e.g., cyclophosphamide, ifosfamide, mafosfamide, and trofosfamide]), nitrosoureas (e.g., carmustine, fotemustine, lomustine, nimustine, N-nitroso-N-methylurea, ranimustine, semustine and streptozotocin), platinum-containing compounds (e.g., cisplatin, carboplatin and oxaliplatin), alkylsulfonates (e.g., busulfan, mannosulfan and treosulfan), hydrazines (e.g., dacarbazine and procarbazine), imidazotetrazines (e.g., mitozolomide and temozolomide), and triazines (e.g., hexamethylmelamine [altretamine]);
ii) cytotoxic antibiotics, such as anthracyclines (e.g., aclarubicin, daunorubicin, doxorubicin, epirubicin, idarubicin, mitoxantrone, pirarubicin and valrubicin), actinomycins (e.g., actinomycin D), bleomycins (e.g., bleomycins A₂ and B₂), mitomycins (e.g., mitomycin C), and plicamycins;
iii) anti-metabolites, such as anti-folates (e.g., aminopterin, methotrexate, pemetrexed and pralatrexate), deoxynucleoside analogs (e.g., 5-azacytidine [azacitidine], 5-aza-2′-deoxycytidine [decitabine], cladribine, clofarabine, cytarabine, decitabine, fludarabine, gemcitabine, nelarabine and pentostatin), fluoropyrimidines (e.g., 5-fluorouracil, 1-hexylcarbamoyl-5-fluorouracil [carmofur], capecitabine, 5-fluoro-5′-deoxyuridine [doxifluridine] and trifluridine), and thiopurines (e.g., thioguanine, azathioprine and 6-mercaptopurine);
iv) anti-microtubule agents, such as dolastatins (e.g., dolastatin 15), epothilones (e.g., epothilones A-F), halichondrins (e.g., halichondrin B) and analogs thereof (e.g., eribulin), maytansine, maytansinoids (e.g., ansamitocin, emtansine, mertansine, ravtansine and soravtansine), taxanes (e.g., paclitaxel, docetaxel and cabazitaxel), vinca alkaloids (e.g., vinblastine, vincristine, vindesine, vinflunine and vinorelbine), colchicine, nocodazole, podophyllotoxin and rhizoxin;
v) histone deacetylase inhibitors, such as trichostatins (e.g., trichostatin A), romidepsin, panobinostat and vorinostat;
vi) kinase inhibitors, such as bortezomib, erlotinib, gefitinib, imatinib, vemurafenib, vismodegib, curcumin, cyclocreatine, deguelin, fostriecin, hispidin, staurosporine and derivatives thereof (e.g., midostaurin), and tyrphostins (e.g., tyrphostins AG 34 and AG 879);
vii) topoisomerase I inhibitors, such as camptothecin, irinotecan and topotecan;
viii) topoisomerase II-targeting agents, such as topoisomerase II poisons (e.g., etoposide, tafluposide, teniposide, doxorubicin and mitoxantrone) and topoisomerase II inhibitors (e.g., novobiocin, merbarone and aclarubicin);
ix) DNA or RNA synthesis inhibitors, such as 3-amino-1,2,4-benzotriazine 1,4-dioxide, cytosine β-D-arabinofuranoside, 5,6-dichlorobenzimidazole 1-β-D-ribofuranoside, ganciclovir and hydroxyurea;
x) protein synthesis inhibitors, such as homoharringtonine;
xi) cell growth and differentiation regulators, such as retinoids (e.g., all-trans retinol [vitamin A], 11-cis retinol, all-trans retinal [vitamin A aldehyde], 11-cis retinal, all-trans retinoic acid [tretinoin], 9-cis-retinoic acid [alitretinoin], 11-cis retinoic acid, 13-cis-retinoic acid [isotretinoin], all-trans retinyl esters, etretinate, acitretin, adapalene, bexarotene and tazarotene);
xii) cell proliferation inhibitors, such as mTOR inhibitors (e.g., everolimus, novolimus, ridaforolimus, sirolimus [rapamycin], temsirolimus, umirolimus [biolimus A9] and zotarolimus), apigenin, cholecalciferol (vitamin D₃) and sex hormone-binding globulin;
xiii) apoptosis inducers, such as 17-allylamino-17-demethoxygeldanamycin, melatonin, mevinolin, psoralen, thapsigargin, troglitazone, inhibitors of histone deacetylases (e.g., romidepsin), and agonists of retinoid X receptors (RXRs) (e.g., retinoids such as bexarotene); and analogs, derivatives and salts thereof,
2) agents that stimulate the immune system, including without limitation:
i) agonists/activators of tumor necrosis factor receptor superfamily member 4 (TNFRSF4, OX40 or CD134), such as OX40-targeting antibodies (e.g., MEDI-6469 and 9B12) and ligands for OX40 (e.g., OX40L);
ii) agonists/activators of TNFRSF member 5 (TNFRSF5 or CD40), such as CD40-targeting antibodies (e.g., dacetuzumab and CP-870,893) and ligands for CD40 (e.g., CD40L [CD154]);
iii) agonists/activators of TNFRSF member 9 (TNFRSF9, 4-1BB or CD137), such as 4-1BB-targeting antibodies (e.g., urelumab [BMS-663513] and PF-05082566) and ligands for 4-1BB (e.g., 4-1BBL);
iv) agonists/activators of TNFRSF member 18 (TNFRSF18, glucocorticoid-induced TNFR-related protein [GITR] or CD357), such as GITR-targeting antibodies (e.g., DTA-1 and TRX518) and ligands for GITR (e.g., GITRL);
v) agonists/activators of toll-like receptors (TLRs), such as ligands for TLR9 (e.g., unmethylated CpG oligodeoxynucleotides [CpG ODNs], such as agatolimod);
vi) cytokines and hormones that stimulate immune cells, such as interleukin-6 (IL-6) and epinephrine (stimulator of, e.g., natural killer cells); and analogs, derivatives, fragments and salts thereof,
3) agents that block immune checkpoints, including without limitation:
i) inhibitors of programmed cell death 1 (PD-1) receptor or ligands thereof (e.g., PD-L1 and PD-L2), such as anti-PD-1 antibodies (e.g., cemiplimab [REGN-2810], nivolumab [BMS-936558, MDX-1106 or ONO-4538], pembrolizumab [lambrolizumab or MK-3475], pidilizumab [CT-011] and MEDI-0680 [AMP-514]), anti-PD-1 fusion proteins (e.g., AMP-224 [containing an F_{c} Ab domain and PD-L2]), anti-PD-L1 antibodies (e.g., avelumab [MSB0010718C], atezolizumab [MPDL3280A or RG7446], durvalumab [MEDI-4736], BMS-936559 [MDX-1105], LY3300054 and YW243.55.S70), and small-molecule inhibitors of PD-L1 (e.g., BMS-1001 and BMS-1166);
ii) inhibitors of cytotoxic T lymphocyte-associated protein 4 (CTLA-4) receptor or ligands thereof, such as anti-CTLA-4 antibodies (e.g., ipilimumab and tremelimumab);
iii) inhibitors of killer cell immunoglobulin-like receptors (KIRs) or ligands thereof, such as anti-KIR antibodies (e.g., lirilumab [IPH2102 or BMS-986015]);
iv) inhibitors of lymphocyte activation gene 3 (LAG-3) receptor or ligands thereof, such as anti-LAG-3 antibodies (e.g., relatlimab [BMS-986016] and GSK2831781);
v) inhibitors of T-cell immunoglobulin and mucin domain-containing 3 (TIM-3, also called hepatitis A virus cellular receptor 2 [HAVCR2]), such as anti-TIM3 antibodies (e.g., LY3321367, MBG453 and TSR-022);
vi) inhibitors of indoleamine 2,3-dioxygenase (IDO or IDO1), such as indoximod (1-methyl-D-tryptophan or NLG-8189), navoximod (GDC-0919), α-methyl-tryptophan, β-carboline (9H-pyrido[3,4-b]indole or norharmane), epacadostat (INCB024360), BMS-986205, NLG-919, and cyclooxygenase 2 (COX-2) inhibitors (e.g., coxibs [such as apricoxib, celecoxib, etoricoxib, lumiracoxib, parecoxib, rofecoxib and valdecoxib], which down-regulate the expression of IDO); and analogs, derivatives, fragments and salts thereof,
4) angiogenesis inhibitors, including without limitation inhibitors of vascular endothelial growth factors (VEGFs) (e.g., squalamine and anti-VEGF antibodies such as bevacizumab) or receptors therefor (VEGFRs) (e.g., axitinib, fruquintinib, pazopanib, regorafenib, sorafenib and sunitinib), inhibitors of platelet-derived growth factors (PDGFs) (e.g., squalamine) or receptors therefor (PDGFRs) (e.g., axitinib, imatinib, nilotinib, pazopanib, sorafenib and sunitinib), inhibitors of fibroblast growth factors (FGFs) (e.g., squalamine) or receptors therefor (FGFRs) (e.g., erdafitinib, pazopanib and anti-FGFR antibodies and fragments thereof), inhibitors of angiopoietins (e.g., anti-angiopoietin antibodies such as nesvacumab and AMG 780) or receptors therefor, inhibitors of integrins (e.g., ALG-1001 and JSM-6427), anecortave (anecortave acetate), angiostatin (e.g., angiostatin K1-3), αvβ₃ inhibitors (e.g., etaracizumab), berberine, bleomycins, borrelidin, carboxyamidotriazole, cartilage-derived angiogenesis inhibitors (e.g., chondromodulin I and troponin I), castanospermine, CM101, cyclopropene fatty acids (e.g., sterculic acid), α-difluoromethylornithine, endostatin, everolimus, fumagillin, genistein, interferon-α, interleukin-12, itraconazole, linomide, matrix metalloproteinase (MMP) inhibitors (e.g., batimastat, cipemastat, ilomastat, marimastat, prinomastat, rebimastat, retinoid X receptor agonists (e.g., retinoids such as bexarotene), tanomastat, and tetracyclines [e.g., doxycycline, incyclinide and minocycline]), 2-methoxyestradiol, pigment epithelium-derived factor (PEDF), platelet factor-4, PPAR-γ agonists (e.g., thiazolidinediones, such as ciglitazone, lobeglitazone, netoglitazone, pioglitazone, rivoglitazone, rosiglitazone and troglitazone), prolactin, sphingosine-1-phosphate inhibitors (e.g., sonepcizumab), squalene, staurosporine, angiostatic steroids (e.g., tetrahydrocortisol) plus heparin, stilbenoids, suramin, SU5416, tasquinimod, tecogalan, tetrathiomolybdate, thalidomide and derivatives thereof (e.g., lenalidomide and pomalidomide), thiabendazole, thrombospondins (e.g., thrombospondin 1), TNP-470, tranilast, Withaferin A, and analogs, derivatives, fragments and salts thereof; and
5) other kinds of antitumor/anticancer agents, including without limitation:
i) inhibitors of class IA phosphoinositide 3-kinase p110α (PI3K-α), such as alpelisib, buparlisib (pan-PI3K), copanlisib (PI3K-α/δ), pictilisib (pan-PI3K), taselisib, voxtalisib (pan-PI3K), GNE-477, INK-1117, PWT-33597, SF-1126 (pan-PI3K) and ZSTK-474;
ii) drug-efflux pump inhibitors, such as P-glycoprotein inhibitors (e.g., mifepristone and verapamil);
iii) cell adhesion inhibitors, such as cimetidine;
iv) Golgi apparatus disruptors, such as brefeldins (e.g., brefeldin A);
v) ionizing radiation, such as X-ray;
vi) radiopharmaceuticals, such as I131-iodide, I131-MIBG (m-iodobenzylguanidine), Ra223-dichloride, Sm153-EDTMP (ethylenediaminotetramethylenephosphoric acid), and Sr89-chloride;
vii) radiation sensitizers of tumor/cancer cells, such as poly(ADP-ribose) polymerase (PARP) inhibitors (e.g., niraparib, olaparib, rucaparib, talazoparib, veliparib, 4-amino-1,8-naphthalimide, BGB290, CEP9722 and E7016), berberine and indomethacin;
viii) enhancers of cell survival after treatment with cytotoxic drugs or radiation, such as pifithrin-α;
ix) vaccines, such as those that stimulate the immune system to recognize proteins produced by tumor/cancer cells and thereby to attack tumor/cancer cells; and analogs, derivatives and salts thereof.

In some embodiments, the disclosed unnatural peptide assembly self-assembles into micelles and is rapidly taken up by cancer cells, especially when combined with an acute cholesterol-depleting agent (MβCD).

### ABBREVIATIONS

CPDR, CellMask PM Deep Red; chol-azide, cholesteryl-TEG-azide; CLSM, confocal laser scanning microscopy; CMC, critical micelle concentrations; CPZ, chlorpromazine; D-BiP or b, D-4,4′-biphenylalanine; DLS, dynamic light scattering; ER, endoplasmic reticulum; LDLR, low density lipoprotein receptor; MβCD, methyl-β-cyclodextrin; NBD, nitrobenzoxadiazole; Microtubule-associated protein 1 light chain 3 beta, LC3B; PM, plasma membrane; RP-HPLC, reverse phase high performance liquid chromatography; SPPS, solid-phase peptide synthesis; TEM, transmission electron microscopy.

### EXAMPLE

The following example is intended only to illustrate the disclosure. Other synthetic processes, assays, studies, protocols, procedures, methodologies, techniques, reagents and conditions may alternatively be used as appropriate.

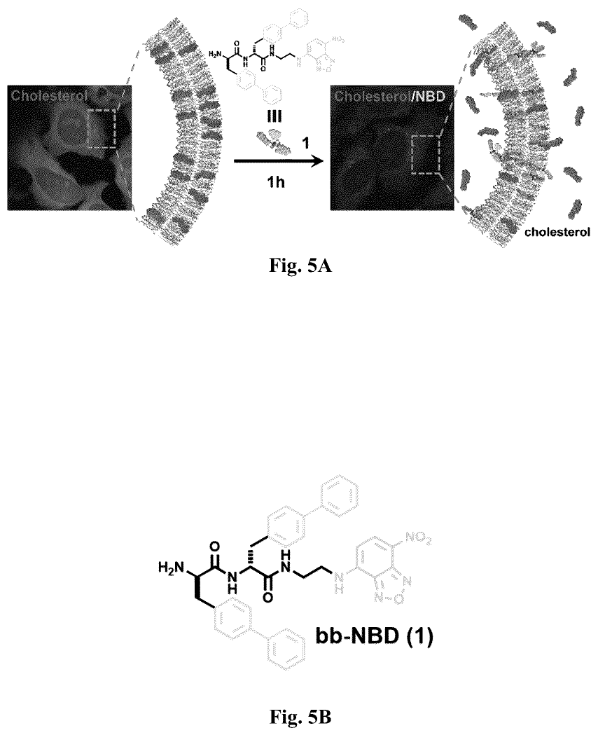

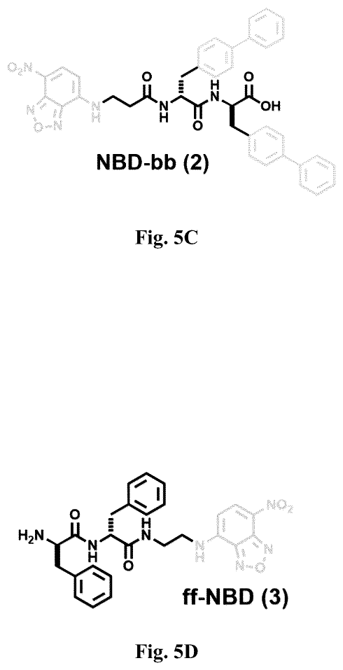

As shown in FIGS. 5A and 5B, during studies of unnatural peptide assemblies, it was discovered that the peptide assemblies deplete cholesterol to inhibit cancer cells. The peptide bb-NBD, containing D-BiP (or b) and C-terminal capped by a fluorophore (NBD), self-assembles to form micelles. bb-NBD enters cells rapidly, and MβCD enhances cellular uptake of bb-NBD. Click reaction reveals that bb-NBD depletes cholesterol from the cell membrane (FIG. 5A). Fluorescent imaging indicates that bb-NBD distributes in membrane-rich organelles, such as ER, Golgi apparatus, and lysosomes. More importantly, bb-NBD potently inhibits malignant cancer cells (e.g., HeLa and PC3), exhibiting synergism with either acute or chronic cholesterol lowering agent. It was also found that C-terminal capping and unnatural amino acid residue (i.e., Bip) are necessary for the observed cholesterol depletion and potent cancer cell inhibition. This work suggests that engineering of unnatural peptide assemblies is a useful methodology to develop molecular platforms that target cell membranes for treating diseases, including cancer.

To enhance the efficacy of peptides for self-assembly, the D-phenylalanine was replaced with an unnatural amino acid, D-BiP, because it possesses enhanced hydrophobicity, and the biphenyl motif appears to favor cellular uptake. This rationale leads to a less explored self-assembling motif, D-BiP-D-BiP or bb. To visualize the peptide assemblies in live cells, NBD was added as a fluorophore, to the C-terminus of bb. This design creates an unnatural peptide, bb-NBD (FIG. 5B). Additionally, two analogs, NBD-bb (FIG. 5C) and ff-NBD (FIG. 5D), were designed as the controls of bb-NBD for examining the roles of the position of the fluorophore and the self-assembling motif, respectively.

SPPS and RP-HPLC yielded purified peptides. CMC values for bb-NBD, NBD-bb, and ff-NBD are 14.8, 373, and 20.6 M, respectively. bb-NBD's lower CMC compared to NBD-bb aligns with protein N-/C-termini pKa (7.7/3.3). TEM revealed nanospheres formed by bb-NBD above CMC (with an average hydrodynamic radius of 103.2±1.9 nm measured by DLS) and is hardly visible below CMC. In contrast, NBD-bb and ff-NBD only form amorphous aggregates above and below their CMCs. These results indicate that a bb motif and an exposed amine group are crucial for bb-NBD's self-assembly in water.

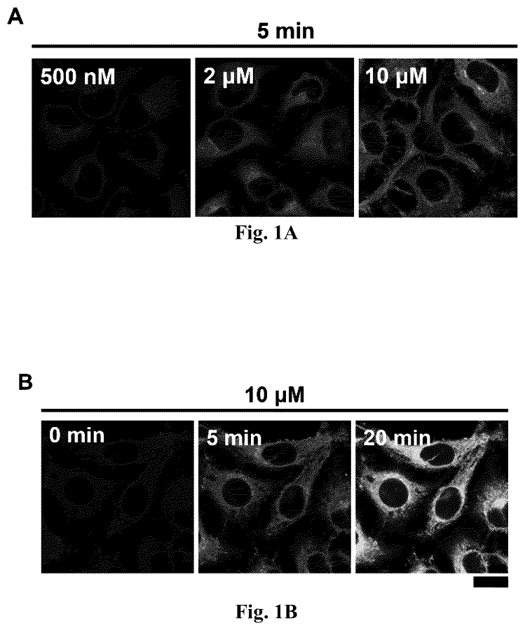

CLSM was used to visualize the cellular uptake of the assemblies of bb-NBD in HeLa cells (FIGS. 1A and 1). The rapid cellular uptake of bb-NBD is both concentration- and time-dependent (FIGS. 1A and 1). For instance, 500 nM of bb-NBD shows noticeable internalization in merely 5 min (FIG. 1A), while 10 mM of bb-NBD gets significantly enriched intracellularly in 20 min (FIG. 1). These results suggest that monomers of bb-NBD (below CMC) insert into cell membranes rapidly, and bb-NBD (at about CMC) likely forms assemblies inside cell membranes to exhibit brighter fluorescence.

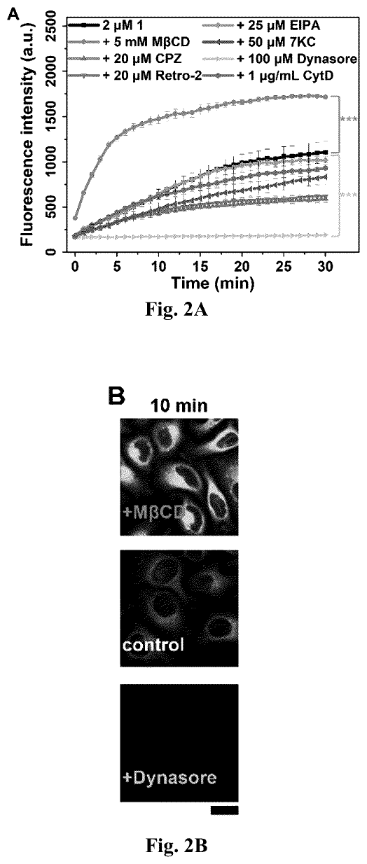

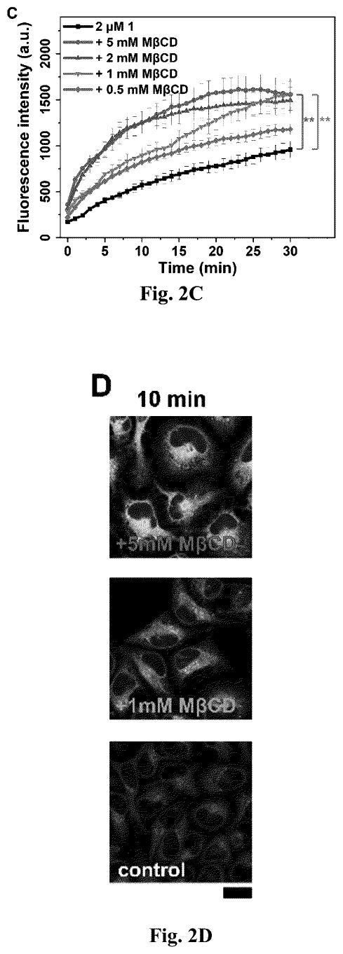

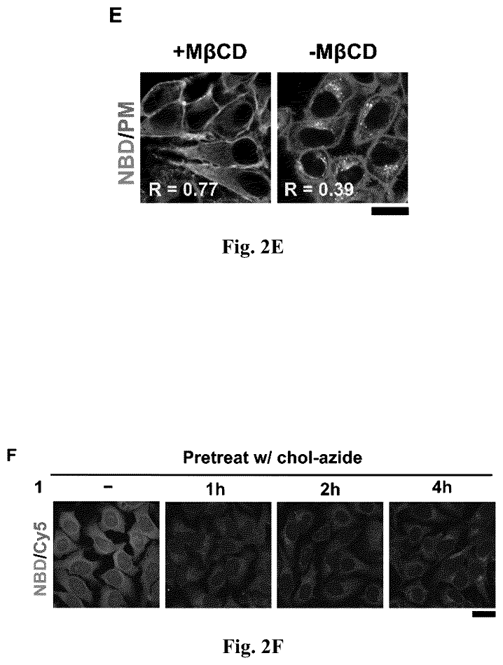

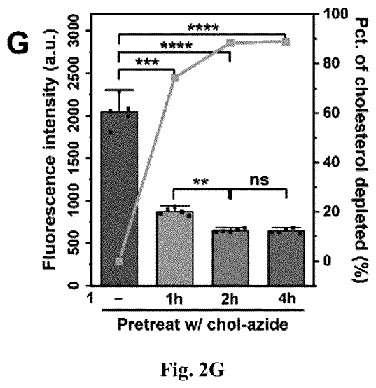

Endocytosis inhibitors were used to provide more insights into the quick internalization of bb-NBD. Dynasore blocks the uptake of bb-NBD (FIG. 2A), suggesting bb-NBD enters cells through an energy-requiring process. In addition, other inhibitors, for example, CPZ and Retro-2 also partially reduce the uptake of bb-NBD (FIG. 2A), indicating a hybrid mode of endocytosis. However, MβCD, a common inhibitor of cholesterol-dependent endocytosis, unexpectedly enhances the uptake of bb-NBD significantly (FIG. 2A). The intracellular fluorescence intensity of bb-NBD in the MβCD treated group is over two times higher than that of the control group in 10 min (FIG. 2B). Single-cell analysis confirms that increasing MβCD concentration boosts the uptake of bb-NBD (FIG. 2C). For example, 1 mM of MβCD increases bb-NBD's uptake by 1.6-fold in 10 min, while 5 mM of MβCD increases it by 2.2-fold (FIG. 2D). Since MβCD removes cellular cholesterol, it is believed that bb-NBD enters cells to fill these vacancies (FIG. 2C).

MβCD also influences the translocation of bb-NBD, evidenced by more green fluorescence on the membrane region compared to the group without MCD. The cells were stained with a PM dye, CPDR, to better visualize the distribution of bb-NBD under cholesterol depletion conditions and verify the translocation. The fluorescence of CPDR is much weaker in the MβCD treated group compared to the control group, implying the successful depletion of cholesterol. After the addition of bb-NBD, strong green fluorescence appears instantaneously at the PM region, with a good colocalization with CPDR (FIG. 2E). In contrast, bb-NBD mainly localizes in the intracellular region in the control group, with a poor colocalization with PM (FIG. 2E). Furthermore, quantifying green fluorescence on the PM region shows that 60% of bb-NBD is present on PM in the MβCD treated group immediately after the addition of bb-NBD, in comparison to 33% in the control group. Although bb-NBD distributes from membrane to cytoplasm over time in both groups, the MβCD treated group retains significantly more bb-NBD on PM—about 45%. Since the PM is rich in cellular cholesterol, these results suggest that after MβCD acutely depletes cholesterol, bb-NBD preferentially assembles at former cholesterol-rich membrane regions. This implies bb-NBD has a high affinity for cholesterol insertion sites under depletion conditions.

Knowing that bb-NBD exhibits a strong affinity for cholesterol-rich membranes, particularly when cholesterol is depleted by MβCD, the bioorthogonal CuAAC click reaction was employed to examine the potential competition between bb-NBD and cholesterol itself. After MβCD depleted endogenous cholesterol, cells were co-incubated with bb-NBD and Cholesterol-azide (chol-azide), as a membrane insertion competitor. CLSM after click reaction with Cy5 alkyne showed minimal Cy5 fluorescence in co-incubated cells (1+chol-azide) compared to the strong signal with chol-azide alone. This suggests bb-NBD preferentially binds to vacated cholesterol sites upon depletion.

bb-NBD was tested whether displaces cholesterol without MβCD. Cells pretreated with chol-azide were incubated with bb-NBD, then visualized. CLSM confirmed that bb-NBD reduces cholesterol (FIG. 2F). Remarkably, bb-NBD induces a 74% and 88% cholesterol depletion within 1 h and 2 h, respectively (FIG. 2G). While depletion remained significant up to 8 h, it reached 40% after 24 h due to intracellular aggregation of bb-NBD. In contrast, a parallel experiment where cells were pretreated with bb-NBD demonstrated that subsequent addition of chol-azide was unable to displace bb-NBD from the cell membranes. These findings suggest that bb-NBD not only has a stronger affinity to vacant cholesterol insertion sites than cholesterol itself but can also directly insert into membranes and replace cholesterol, demonstrating the unique cholesterol depletion action of bb-NBD. Furthermore, whether MβCD could remove bb-NBD from cell membranes, as it does with cholesterol, was tested. After pretreating HeLa cells with bb-NBD for 1 h, MβCD was added to the cells for another 30 min. Compared to cells treated with only bb-NBD for 1.5 h, MβCD treated cells show no significant changes in the fluorescence intensity of bb-NBD, further confirming the strong affinity of bb-NBD to cell membranes. However, immunostaining of LDLR (low-density lipoprotein receptor), which mediates the endocytosis of cholesterol, shows an inverse relationship between bb-NBD and LDLR, indicating LDLR-independent internalization of bb-NBD.

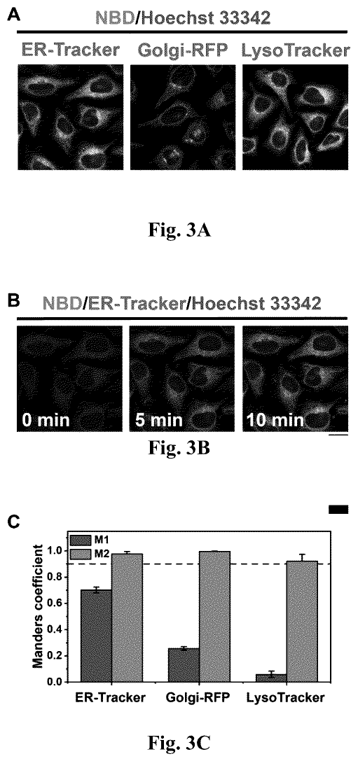

To better understand the intracellular distribution of bb-NBD, various trackers or transfection methods were used to stain different organelles in HeLa cells. This was done to analyze co-localization with bb-NBD using CLSM. When the cells were treated with bb-NBD for 20 min, significant overlaps of its green fluorescence with the ER, Golgi apparatus and lysosomes were observed. This was evidenced by high M2 values (fraction of organelle overlapping bb-NBD) of over 0.9 (FIG. 3A and FIG. 3C). Time-lapse CLSM images demonstrate the rapid targeting of these organelles by bb-NBD, with noticeable colocalization within 5 min and pronounced enrichment at ER within 10 min (FIG. 3B). Given that these membrane-bound organelles are crucial for cholesterol transport and biosynthesis, the findings imply that it has a strong affinity for intracellular cholesterol-rich membranes, in addition to the PM.

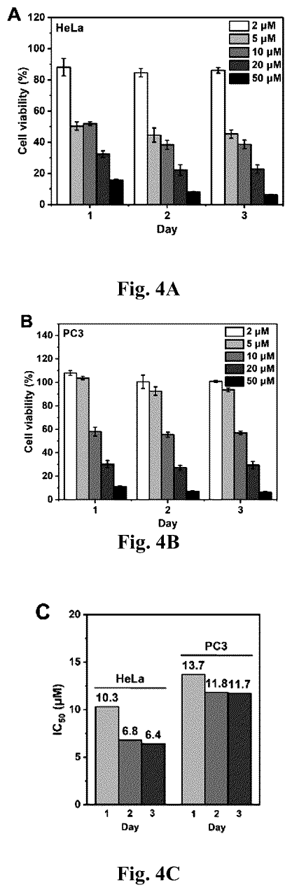

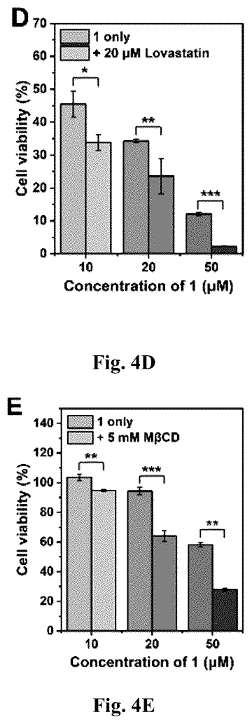

Based on the unique cholesterol depletion behavior of bb-NBD, its cytotoxicity against HeLa and PC3 cells was tested. The MTT assay revealed that bb-NBD exhibits strong cytotoxicity against both cell lines, with IC50 values of 10.3 and 13.7 PM, respectively (FIGS. 4A-C). Furthermore, the cytotoxicity of bb-NBD increases with time (FIG. 4C), suggesting that it inhibits cancer cells related to its intracellular accumulation. Moreover, bb-NBD exhibits synergism with other cholesterol depletion agents. Co-incubating bb-NBD with lovastatin and MβCD, which deplete cholesterol by inhibiting cholesterol biosynthesis and cholesterol extraction, respectively, significantly decreases cell viability in a concentration-dependent manner (FIGS. 4D and 4E). Given the enhanced cellular uptake under cholesterol depletion conditions, these results suggest that the cytotoxicity induced by bb-NBD is positively correlated to its intracellular accumulation.

To elucidate the cell death pathways triggered by bb-NBD, a series of cell death inhibitors were employed. The results indicate bb-NBD induces cell death through multiple pathways, including apoptosis, necrosis, and oncosis. Further investigation using lysosomal staining reveals that bb-NBD induces enlargement of lysosomes. Additionally, immunostaining demonstrates a concentration-dependent increase in LC3B expression. Western blot analysis of LC3 also clearly reveals its upregulation induced by bb-NBD. These combined observations suggest autophagic cell death.

A comparison was conducted on compounds (bb-NBD, NBD-bb, and ff-NBD) for their cholesterol depletion in HeLa cells. CLSM shows that NBD-bb, possessing a carboxylate C-terminus, localizes to the PM exterior, supporting the bb motif's membrane targeting. Unlike bb-NBD, NBD-bb's poor uptake (likely due to its C-terminal negative charges) results in low cytotoxicity (IC50>200 μM). Conversely, ff-NBD, with an ff motif, shows rapid uptake, suggesting a diffusion mechanism different from bb-NBD. Inhibitor studies further confirm this: neither MβCD nor dynasore significantly affects ff-NBD's uptake. Although MβCD boosts the uptake of bb-NBD by more than 2-fold in 5 min, it has a minimal impact on ff-NBD, confirming the bb motif's exclusive cholesterol depletion function. Despite fast uptake, ff-NBD exhibits mild cytotoxicity against HeLa cells due to its lack of cholesterol targeting ff-NBD.

Click chemistry has confirmed unnatural peptide assemblies deplete cell membrane cholesterol. The unnatural peptide assemblies localize in membrane-rich organelles, including the endoplasmic reticulum, Golgi apparatus, and lysosomes. Furthermore, unnatural peptide assemblies potently inhibit malignant cancer cells, working synergistically with cholesterol-lowering agents. Control experiments have confirmed that C-terminal capping and unnatural amino acid residue (i.e., Bip) are essential for both cholesterol depletion and potent cancer cell inhibition. Accordingly, the disclosed unnatural peptide assemblies are a promising platform for targeting cell membranes in controlling cell fates.

A family of unnatural peptide assemblies for modulating membrane biophysics for therapeutic purposes was discovered. The unnatural peptide assemblies typically contain an unnatural lipophilic motif, for example, D-biphenylalanine-D-biphenylalanine, for self-assembly, and a fluorophore for imaging. More specifically, 2 μM of bb-NBD was found to deplete approximately 75% of cholesterol from cancer cells in one hour. Furthermore, it was found that the unnatural peptide assemblies could potently inhibit the proliferation of cancer cells with the IC₅₀ ranging from submicromolar to about 10 μM.

The IC₅₀s of some embodiments of the unnatural peptide assemblies of Formula I are provided in the Table 1. As shown in Table 1, the peptide assemblies can potently inhibit the proliferation of cancer cells, with the IC₅₀ ranging from submicromolar to about 10 μM.

TABLE 1*

| Cell | | | | IC50 |
|---|---|---|---|---|
| Lines | X-group | X₁-group | X₂-group | (nM) |
| HeLa | X = NH₂ | biphenyl | biphenyl | 10300 |
| cells | | | | |
| HeLa | X = OH | biphenyl | biphenyl | 630 |
| cells | | | | |
| HeLa cells | 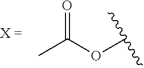 [SMILES: *OC(C)=O] | biphenyl | biphenyl | 980 |
| HeLa cells | 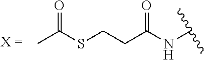 [SMILES: *NC(=O)CCSC(C)=O] | biphenyl | biphenyl | 340 |
| HeLa | X = NHX₃, X₃ = Glycine | biphenyl | biphenyl | 500 |
| cells | | | | |

*Y is 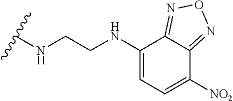 [SMILES: *NCCNc1ccc([N+](=O)[O-])c2nonc12]
for all compounds in Table 1.

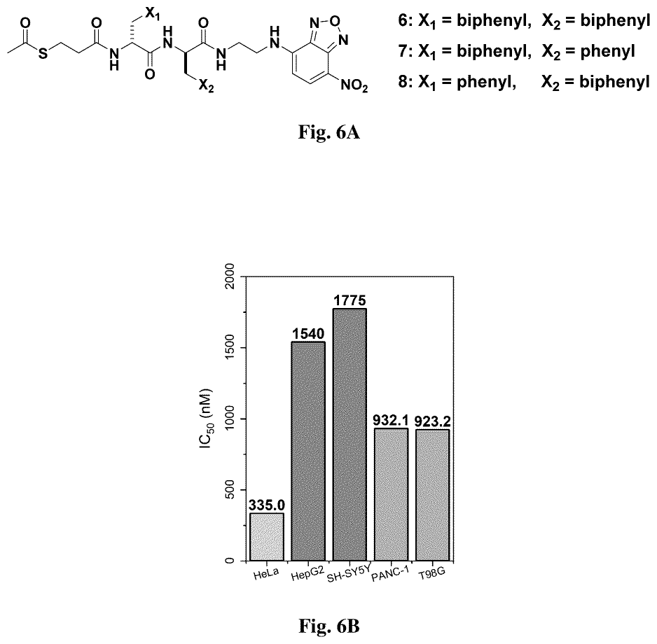

As shown in FIGS. 6A and 6B, when X₁ is biphenyl and X₂ is biphenyl, the unnatural peptide assembly (peptide 6) has the potential to interfere with cellular trafficking by modulating membrane biophysics. Preliminary results indicate that peptide 6 could form intracellular vesicles, sequestering endosomal proteins and consequently disrupting endosomal recycling to kill cancer cells. Furthermore, two derivative peptides 7 and 8, featuring distinct self-assembly motifs, exhibit similar characteristics.

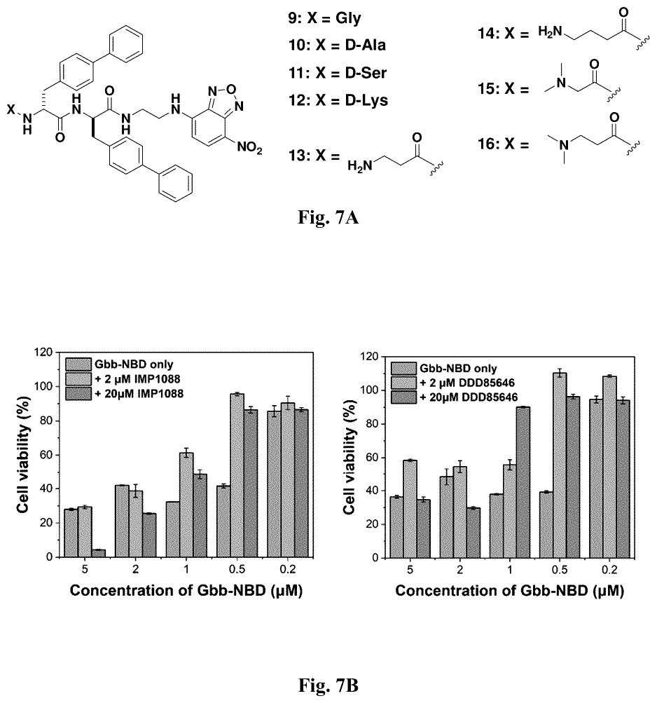

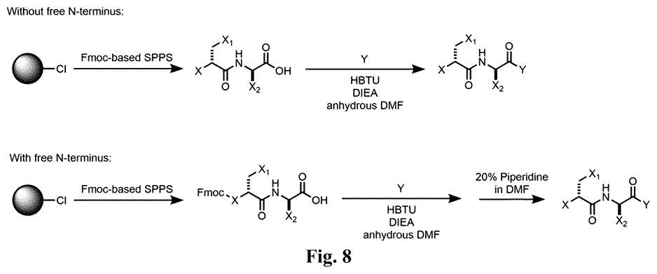

Building on the exploration of unnatural peptides modulating cell membranes, the feasibility of using an unnatural self-assembly motif to target membrane-bound enzymes was assessed. As shown in FIGS. 7A and 7B, peptide 9, which incorporates an additional glycine residue at its N-terminus, is a potential substrate for N-myristoyltransferase (NMT). Preliminary results indicate that the high cytotoxicity of peptide 9 against HeLa cells can be almost fully rescued by NMT inhibitors, IMP1088 and DDD85646 (FIG. 7B). Confocal imaging also verified that pretreatment with these inhibitors decreases the cellular uptake of peptide 9. Additionally, peptides 10-16 were developed, each featuring distinct N-terminal residues, to facilitate comparative analysis.

### REFERENCES

The following references are incorporated herein by reference in their entirety for all purposes.
1. Zhang, Q.; Tan, W.; Liu, Z.; Zhang, Y.; Wei, W. S.; Fraden, S.; Xu, B. Unnatural Peptide Assemblies Rapidly Deplete Cholesterol and Potently Inhibit Cancer Cells. J Am Chem Soc.; 2024 May 15; 146(19):12901-12906
2. Silvente-Poirot, S.; Poirot, M. Cholesterol metabolism and cancer: the good, the bad and the ugly. Curr. Opin. Pharm. 2012, 6 (12), 673-676.
3. Kolb, H. C.; Finn, M.; Sharpless, K. B. Click chemistry: diverse chemical function from a few good reactions. Angew. Chem. Int. Ed. 2001, 40 (11), 2004-2021.
4. Zidovetzki, R.; Levitan, I. Use of cyclodextrins to manipulate plasma membrane cholesterol content: evidence, misconceptions and control strategies. BBA-Biomembranes 2007, 1768 (6), 1311-1324.
5. Wang, H.; Song, Y.; Wang, W.; Chen, N.; Hu, B.; Liu, X.; Zhang, Z.; Yu, Z. Organelle-Mediated Dissipative Self-Assembly of Peptides in Living Cells. J. Am. Chem. Soc. 2023, 146 (1), 330-341.
6. Liu, X.; Li, M.; Liu, J.; Song, Y.; Hu, B.; Wu, C.; Liu, A. A.; Zhou, H.; Long, J.; Shi, L.; Yu, Z. In situ self-sorting peptide assemblies in living cells for simultaneous organelle targeting. J. Am. Chem. Soc. 2022, 144 (21), 9312-9323.
7. Shi, J.; Fichman, G.; Schneider, J. P. Enzymatic Control of the Conformational Landscape of Self-Assembling Peptides. Angew. Chem. Int. Ed. 2018, 57 (35), 11188-11192.
8. Hughes, M.; Debnath, S.; Knapp, C. W.; Ulijn, R. V. Antimicrobial properties of enzymatically triggered self-assembling aromatic peptide amphiphiles. Biomater. Sci. 2013, 1 (11), 1138-1142.
9. Zhang, Q.; Tan, W.; Ashton-Rickardt, I.; Lau, W.; Zou, L.; Xu, B. Intracellular Peptide N-Myristoylation for Cancer Cell Ferroptosis without Acquired Resistance. J Am Chem Soc.; 2025, 147 (44), 40120-40125.

What is claimed is:

1. An unnatural peptide assembly of Formula I, or a tautomer, a pharmaceutically acceptable salt, a solvate, a hydrate, a clathrate or a polymorph thereof: 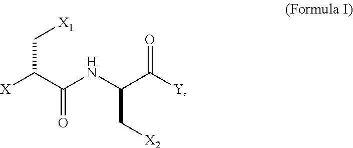
wherein X is selected from NH₂, NHX₃, OH, 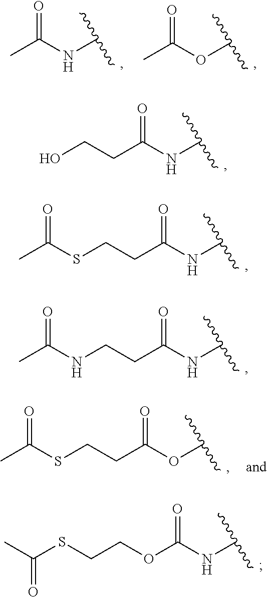
X₁ is selected from biphenyl and phenyl;
X₂ is selected from biphenyl and phenyl;
X₃ is selected from Glycine, D-Alanine, D-Serine, D-Lysine, 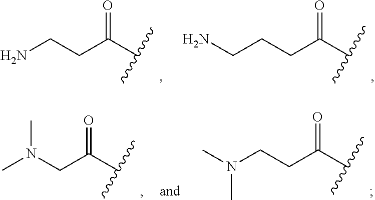
and
Y is selected from

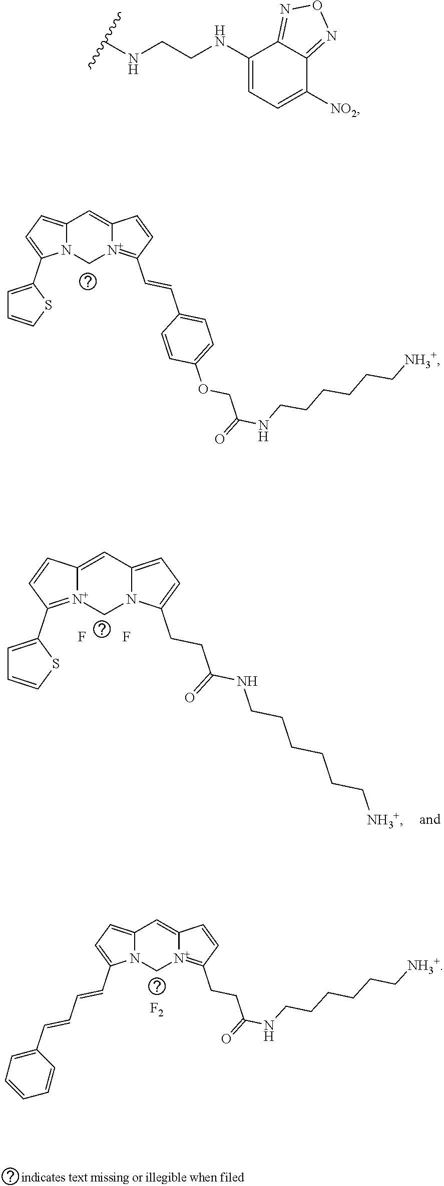

2. The unnatural peptide assembly of claim 1, wherein X is NH₂, X₁ is biphenyl, X₂ is biphenyl, and Y is

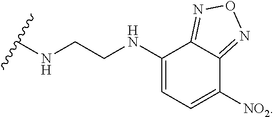 [SMILES: *NCCNc1ccc([N+](=O)[O-])c2nonc12]

3. The unnatural peptide assembly of claim 1, wherein X is 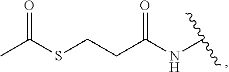 [SMILES: *NC(=O)CCSC(C)=O]
X₁ is biphenyl, X₂ is biphenyl, and Y is

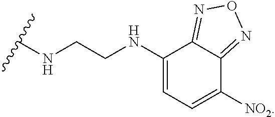 [SMILES: *NCCNc1ccc([N+](=O)[O-])c2nonc12]

4. The unnatural peptide assembly of claim 1, wherein X is NHX₃ and X₃ is Glycine;
wherein X₁ is biphenyl and wherein X₂ is biphenyl; wherein Y is

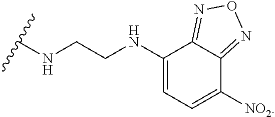 [SMILES: *NCCNc1ccc([N+](=O)[O-])c2nonc12]

5. A pharmaceutical composition comprising the unnatural peptide assembly of claim 1, or a pharmaceutically acceptable salt, a solvate, a hydrate, a clathrate or a polymorph thereof; and one or more pharmaceutically acceptable excipients or carriers.

6. The pharmaceutical composition of claim 5, wherein the unnatural peptide assembly is in an aqueous medium.

7. A method of treating a tumor or a cancer, comprising administering to a subject in need thereof a therapeutically effective amount of an unnatural peptide assembly of claim 1, or a tautomer, a pharmaceutically acceptable salt, a solvate, a hydrate, a clathrate or a polymorph thereof, to treat the tumor or the cancer.

8. The method of claim 7, wherein the unnatural peptide assembly is administered orally.

9. The method of claim 7, wherein the unnatural peptide assembly is administered intratumorally, parenterally, intravenously, subcutaneously or intramuscularly.

10. A method of inhibiting the proliferation or growth of cancer cells comprising contacting cancer cells, with the unnatural peptide assembly of claim 1, or a tautomer, a pharmaceutically acceptable salt, a solvate, a hydrate, a clathrate or a polymorph thereof.

11. The method of claim 10, wherein the contacting occurs in vivo, ex vivo or in vitro.

12. The method of claim 10, wherein the cancer cells are cervical cancer cells, prostate cancer cells, or liver cancer cells.

13. An unnatural peptide assembly of Formula II, or a tautomer, a pharmaceutically acceptable salt, a solvate, a hydrate, a clathrate or a polymorph thereof:

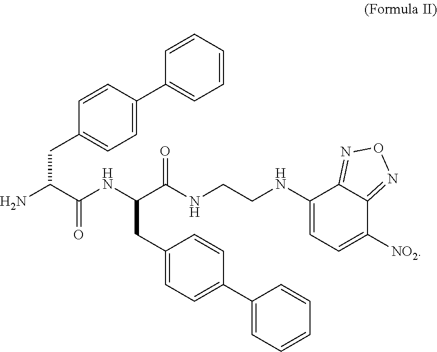 [SMILES: N[C@H](Cc1ccc(-c2ccccc2)cc1)C(=O)N[C@H](Cc1ccc(-c2ccccc2)cc1)C(=O)NCCNc1ccc([N+](=O)[O-])c2nonc12]

14. An unnatural peptide assembly of Formula III, or a tautomer, a pharmaceutically acceptable salt, a solvate, a hydrate, a clathrate or a polymorph thereof:

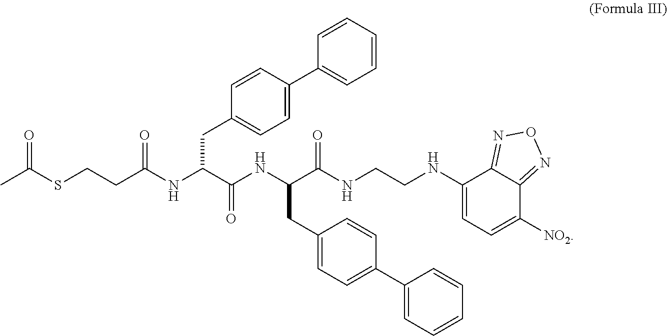 [SMILES: CC(=O)SCCC(=O)N[C@H](Cc1ccc(-c2ccccc2)cc1)C(=O)N[C@H](Cc1ccc(-c2ccccc2)cc1)C(=O)NCCNc1ccc([N+](=O)[O-])c2nonc12]

15. An unnatural peptide assembly of Formula IV, or a tautomer, a pharmaceutically acceptable salt, a solvate, a hydrate, a clathrate or a polymorph thereof:

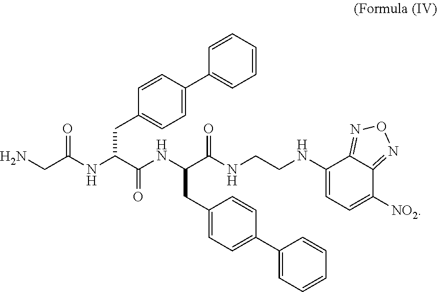 [SMILES: NCC(=O)N[C@H](Cc1ccc(-c2ccccc2)cc1)C(=O)N[C@H](Cc1ccc(-c2ccccc2)cc1)C(=O)NCCNc1ccc([N+](=O)[O-])c2nonc12]

16. A pharmaceutical composition comprising the unnatural peptide assembly of claim 1, or a tautomer, a pharmaceutically acceptable salt, a solvate, a hydrate, a clathrate or a polymorph thereof; and one or more pharmaceutically acceptable excipients or carriers.

17. The pharmaceutical composition of claim 16, wherein the unnatural peptide assembly is in an aqueous medium.

18. A method of treating a tumor or cancer, comprising administering to a subject in need thereof a therapeutically effective amount of the pharmaceutical composition of claim 16, or a tautomer, a pharmaceutically acceptable salt, a solvate, a hydrate, a clathrate or a polymorph thereof, to treat a tumor or cancer.

19. A method of inhibiting the proliferation or growth of cancer cells comprising contacting cancer cells, with the pharmaceutical composition of claim 16, or a tautomer, a pharmaceutically acceptable salt, a solvate, a hydrate, a clathrate or a polymorph thereof.

20. The method of claim 19, wherein the contacting occurs in vivo, ex vivo or in vitro.
